SEQUENCE LISTING (33 sequences)

SEQ ID NO: 1 - protein, 29 aa, synthetic construct
FWVLVVVGGVLACYSLLVTVAFIIFWVRS

SEQ ID NO: 2 - protein, 70 aa, synthetic construct
NGVTVSSALSNSIMYFSHFVPVFLPAKPTTTPAPRPPTPAPTIASQPLSLRPEACRPAAGGAVHTRGLDP

SEQ ID NO: 3 - protein, 10 aa, synthetic construct
EQKLISEEDL

SEQ ID NO: 4 - protein, 39 aa, synthetic construct
KRSRLLHSDYMNMTPRRPGPTRKHYQPYAPPRDFAAYRS

SEQ ID NO: 5 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 176 aa, synthetic construct
AGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 7 - protein, 356 aa, synthetic construct
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 8 - protein, 244 aa, synthetic construct
FWVLVVVGGVLACYSLLVTVAFIIFWVRSKRSRLLHSDYMNMTPRRPGPTRKHYQPYAPPRDFAAYRSAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 9 - protein, 317 aa, synthetic construct
FWVLVVVGGVLACYSLLVTVAFIIFWVRSAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 261 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEI

SEQ ID NO: 11 - protein, 697 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 12 - protein, 585 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRLLHSDYMNMTPRRPGPTRKHYQPYAPPRDFAAYRSAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 13 - protein, 662 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 521 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 374 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSR

SEQ ID NO: 16 - protein, 547 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 17 - protein, 697 aa, synthetic construct; identical to SEQ ID NO: 11

SEQ ID NO: 18 - protein, 698 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEAARPAAGGAVHTRGLDPFWLVLILLTFVLFVFILYWVITWYLIWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 19 - protein, 697 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEAARPAAGGAVHTRGLDPFWLLFILVAILGGLFGAIVAFLLALWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 20 - protein, 696 aa, synthetic construct
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEAARPAAGGAVHTRGLDPFWLLVALLALLAVIAALLAAIFALWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 21 - protein, 9 aa, synthetic construct
YPYDVPDYA

SEQ ID NO: 22 - protein, 6 aa, synthetic construct
HHHHHH

SEQ ID NO: 23 - protein, 8 aa, synthetic construct
DYKDDDDK

SEQ ID NO: 24 - protein, 6 aa, synthetic construct
DTYRYI

SEQ ID NO: 25 - protein, 6 aa, synthetic construct
TDFYLK

SEQ ID NO: 26 - protein, 6 aa, synthetic construct
EYMPME

SEQ ID NO: 27 - protein, 14 aa, synthetic construct
SGFANELGPRLMGK

SEQ ID NO: 28 - protein, 11 aa, synthetic construct
MASMTGGQQMG

SEQ ID NO: 29 - protein, 14 aa, synthetic construct
GKPIPNPLLGLDST

SEQ ID NO: 30 - protein, 13 aa, synthetic construct
GAPVPYPDPLEPR

SEQ ID NO: 31 - protein, 15 aa, synthetic construct
KETAAAKFERQHMDS

SEQ ID NO: 32 - protein, 16 aa, synthetic construct
CGLNDIFEAQKIEWHE

SEQ ID NO: 33 - protein, 12 aa, synthetic construct
SQPELAPEDPED

(19) United States
(12) Patent Application Publication
(10) Pub. No.: US 2026/0144873 A1
(43) Pub. Date: May 28, 2026

(54) CELL THERAPY

(71) Applicant: THE WALTER AND ELIZA HALL INSTITUTE OF MEDICAL RESEARCH, Melbourne (AU)

(72) Inventors: Stephen NUTT, Melbourne (AU); Shengbo ZHANG, Melbourne (AU); Michael CHOPIN, Melbourne (AU)

(21) Appl. No.: 19/121,580

(22) PCT Filed: Oct. 17, 2023
(86) PCT No.: PCT/AU2023/051023

§ 371 Date: Apr. 16, 2025

(30) Foreign Application Priority Data
Oct. 17, 2022 (AU) 2022903034

(51) Int. Cl. A61K 40/31; A61K 35/15; A61K 40/11; A61K 40/42; A61P 37/04; C07K 14/705; C07K 14/715; C07K 14/725; C07K 16/32; C12N 5/0784

(52) U.S. Cl. CPC A61K 40/31; A61K 35/15; A61K 40/11; A61K 40/4205; A61P 37/04; C07K 14/7051; C07K 14/70517; C07K 14/70521; C07K 14/7155; C07K 16/32; C12N 5/0639; A61K 2239/13; A61K 2239/21; A61K 2239/22; C07K 2317/622; C12N 2501/26; C12N 2510/00

(57) ABSTRACT

The present invention provides for chimeric antigen receptor constructs capable of being expressed in dendritic cells (DCs), and DCs modified to express one or more chimeric antigen receptors (CARs) as well as compositions comprising these modified DCs and methods of stimulating an adaptive immune response in a subject. The intracellular domain of the CAR comprises a toll-interleukin receptor (TIL) intracellular signalling domain and a costimulating domain selected from CD3 signalling domain, CD28 signalling domain and a combined CD28 and CD3 signalling domain.

### RELATED APPLICATION

The present application claims priority from Australian provisional patent application AU2022903034, filed Oct. 17, 2022, the entire contents of which is incorporated herein by reference.

### REFERENCE TO SEQUENCE LISTING

The entire content of the electronic submission of the sequence listing is incorporated by reference in its entirety for all purposes.

### TECHNICAL FIELD

Disclosed herein are dendritic cells (DCs) modified to express one or more chimeric antigen receptors (CARs) as well as compositions comprising same and methods of generating an adaptive immune response in a subject.

### BACKGROUND

Chimeric antigen receptor (CAR) therapy has achieved great clinical success against haematological malignancies. It is based on synthetic receptors with both antigen recognition and signal transduction functions. The single chain variable fragment (scFv) in a CAR retains its antigen recognition specificity from the variable regions of the heavy and light chains of the monoclonal antibody. Signal transduction of the CAR construct largely depends on the signalling domains of the original immune receptors. However, while CAR-T therapy has proven remarkable successful in treating blood based cancers such as leukemia, its application to solid tumours has been more problematic. For example, CAR-T cells exhibit a remarkable 80-100% clinical response (CR) in end-stage relapsed acute lymphoblastic leukemia (ALL) patients but only a 1% CR in solid tumours. This is due to difficulties in developing CAR-T cells that can access solid tumours since solid tumours are often surrounded by a physical barrier formed by tumour associated stromal cells limiting T cell infiltration, the broad antigen heterogeneity displayed by solid tumours and the highly immunosuppressive microenvironment surrounding some solid tumours. Furthermore, CAR-T cells downregulate the expression of adhesion molecules on their cell surface, limiting their adhesion properties to an already dysregulated tumour vasculature. Adding to these extrinsic factors, intrinsic factors inherent to the CAR-T cells can blunt their efficacy, leading to their exhaustion. In addition, CAR-T cell treatment can lead to toxicities resulting in the destruction of heathy cells including the CAR-T cells themselves. CAR-T therapies have also evoked strong on target off tumour toxicity against healthy tissue in some trials.

Given the above challenges, researchers have also turned their attention to the use of myeloid cells, including monocytes, macrophages and DCs, which can produce a large amount of pro-inflammatory cytokines to activate T cells and might be able to educate the tumour microenvironment to promote anti-tumour immune responses. Some researchers found that CARs carried by adenoviral vectors overcame the inherent resistance of primary human macrophages to genetic manipulation and imparted a sustained pro-inflammatory phenotype. CAR macrophages (CAR-Ms) have demonstrated antigen-specific phagocytosis and tumour clearance in vitro (M Klichinsky et al., (2020) Nature Biotechnology Vol 38:947-953).

Macrophages however have limited capacity to present antigen to T cells as their major function in the tumour context is to phagocytose the dead and dying tumour cells and produce pro-inflammatory or immunosuppressive cytokines depending on the context the dead or dying cell is encountered. This may be a critical limitation of macrophages as a cell therapy as the initiation of effector and memory T cell responses is essential for the tumour clearance and critical in preventing tumour relapse, which is the major cause of death for cancer patients. Getting CARs to express in the cells has also proven to be a challenge because of the non-replicative nature of macrophages (M Mukhopadhyay (2020) Nature Methods 17:561).

Dendritic cells (DCs) are professional antigen-presenting cells that link innate and adaptive immunity and are critical for the induction of protective immune responses against pathogens. Both T and B lymphocytes are key players in mounting an adaptive immune response against the pathogens or malignant cells resulting in the production of effector T cells and high affinity antibodies produced by plasma cells. The adaptive immune system also has the important attribute that antigen-specific T and B cells can differentiate into a population of memory cells that can persist for many years in the host. Upon subsequent exposure to the same pathogen, these cells can rapidly and vigorously respond to the threat, resulting in a quicker and more effective immune response.

DCs recognise, take up and present pathogen-derived antigens to T cells by the Major Histocompatibility Complex (MHC) molecules. DCs also provide the necessary costimulatory signals and pro-inflammatory cytokines, to effectively initiate the adaptive immune response.

Their activity is central to this ability to discriminate the dangerous from the benign and for promoting the optimal type of immune response.

The expression of CARs on monocyte derived dendritic cells (moDCs) has also been explored. Intracellular domain truncated anti-HER2 CAR expression in moDCs was shown to enhance phagocytosis of tumour cell derived exosomes, thereby increasing their antigen presentation. US20200247870 describes use of monocyte derived macrophages generated by differentiating CD14+ selected cells (from normal donor apheresis products) in GM-CSF conditioned media for 7 days. To optimize delivery of CAR via lentiviral transduction, anti-HER2 lentivirus was used to transduce macrophages at different points of the monocyte to macrophage differentiation process.

There is a need in the art for alternative and improved therapies for fighting tumours, in particular semi-solid and solid tumours, while creating an adaptive immune response by harnessing anti-tumour T cells.

### SUMMARY

The present disclosure is based on the generation of modified dendritic cells (DCs) comprising a chimeric antigen receptor (CAR) that exhibit enhanced tumour recognition leading to superior antigen presentation ability and hence a more effective adaptive immune response in vivo. More particularly, the modified DCs exhibit enhanced ability to crosspresent tumour-specific antigens to CD8+ T cells to promote T cells proliferation and cytokine secretion.

In particular, the inventors have successfully generated functional CAR-DCs that comprise a toll-interleukin-1 receptor (TIR) domain, in particular the TIR from toll-like receptor 4 (i.e. TIR4). Furthermore, the inventors found that strong DC pro-inflammatory responses could be obtained when a CD28 signalling domain and/or CD3ζ signalling domain was present in the CAR in combination with TIR4.

In a first aspect, there is provided a chimeric antigen receptor (CAR) construct comprising:
(i) an antigen binding domain;
(ii) a transmembrane domain (TM); and
(iii) an intracellular domain (IC) comprising a toll-interleukin receptor (TIR) intracellular signalling domain and a costimulatory signalling domain selected from a CD3 signalling domain, CD28 signalling domain, and a combined CD28 and CD3 signalling domain;
   wherein the CAR construct is capable of being expressed or is functional in a dendritic cell.

In a second aspect, there is provided a modified dendritic cell comprising a chimeric antigen receptor (CAR) construct, the CAR construct comprising:
(i) an antigen-binding domain;
(ii) a transmembrane domain (TM); and
(iii) an intracellular domain (IC) comprising a toll-interleukin receptor (TIR) intracellular signalling domain; and a costimulatory signalling domain selected from a CD3 signalling domain, CD28 signalling domain and a combined CD28 and CD3 signalling domain;

In one example the TIR intracellular signalling domain is situated after the costimulatory signalling domain, that is it is distal to the costimulatory signalling domain. In a particular example, the TIR is situated after the costimulatory domains so that the order of domains in an N to C orientation is transmembrane domain-costimulatory domain. In a particular example, the TIR is situated after each of the CD28 and CD3 costimulatory domains or situated after the combined CD28 and CD3 costimulatory domains so that the order of domains is in an N to C orientation and the arrangement of the domains is TM-CD3-TIR, or TM-CD28_{IC}-TIR or TM-CD28_{IC}-CD3-TIR or TM-CD3-CD28_{TM}-TIR.

In one example, the transmembrane domain is a CD28 transmembrane domain.

In one particular example, the order of the domains is in an N to C orientation and the arrangement of the domains the CAR construct is CD28_{TM}-CD3-TIR, or CD28_{TM}-CD28_{IC}-TIR or CD28_{TM}-CD28_{IC}-CD3-TIR or CD28_{TM}-CD3-CD28_{TM}-TIR.

In another example, the CD28 transmembrane domain comprises or consists of the sequence of

(SEQ ID NO: 1)
FWVLVWVGGVLACYSLLVTVAFIIFWVRS.

In another example, the transmembrane domain comprises or consists of the sequence of

(SEQ ID NO: 17)
FWLTVALILGIFLGTFIAFWVVYLLWVRS

In one example, the dendritic cell is a conventional type 1 (cDC1) cell. In another example, the cDC1 expresses one or more markers selected from the group consisting of BTLA, CADM1, CD8A, CLEC9A, ITGAE, ITGAX, LY75, THBD (CD141), XCR1, and CD26.

In a third aspect, there is provided a modified dendritic cell comprising one or more nucleic acid sequences encoding a CAR construct comprising:
(i) an antigen binding domain;
(ii) a transmembrane domain (TM); and
(iii) an intracellular domain (IC) comprising a toll-interleukin receptor (TIR) intracellular signalling domain and a costimulatory signalling domain selected from a CD3 signalling domain, CD28 signalling domain, and a combined CD28 and CD3 signalling domain.

In one example, the transmembrane domain is a CD28 transmembrane domain. In another example, the CD28 transmembrane domain comprises or consists of the sequence

(SEQ ID NO: 1)
FWVLVVVGGVLACYSLLVTVAFIIFWVRS.

In another example, the transmembrane domain comprises or consists of the sequence

(SEQ ID NO: 17)
FWLTVALILGIFLGTFIAFWVVYLLWVRS

In some examples, the CAR further comprises a CD8 hinge sequence. In one example, the CD8 hinge sequence comprises or consists of the sequence of the sequence NGVTVSSALSNSIMYFSHFVPVFLPAKPTTTPAPRPPTPAPTIASQPLSLRPEACRPAAGGAVHTRGL (SEQ ID NO:2). In one example, the CD8 hinge is situated before the CD28 transmembrane domain.

In some examples, the CAR further comprises a tag. In one example, the tag is a Myc tag. In another example the tag is located adjacent to, or within the C-terminal sequence of the antigen-binding domain. In another example, the tag is located at the N-terminus to the CD8 hinge sequence. In one example, the tag comprises or consists of the sequence EQKLISEEDL (SEQ ID NO:3).

In one example, the costimulatory signalling domain is a CD28 intracellular domain comprising or consisting of the sequence of

(SEQ ID NO: 4)
KRSRLLHSDYMNMTPRRPGPTRKHYQPYAPPRDFAAYRS.

In one example, the costimulatory signalling domain is a CD3 signalling domain, preferably a CD3ζ domain. In one example, the CD3ζ intracellular costimulatory signalling domain comprises or consists of the sequence of

(SEQ ID NO: 5)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In one example, the TIR intracellular signalling domain is selected from Toll-like receptor 4, Toll-like receptor 3, Toll-like receptor 9, Toll-like receptor 11 and Toll-like receptor 13. In another example, the TIR domain is a TLR4 domain (designated TIR4). In another example, the TIR4 domain comprises or consists of the sequence

(SEQ ID NO: 6)
AGCKKYSRGESIYDAFVIYSSQNEDWVRNELVKNLEEGVPRFHLCLHYR
DFIPGVAIAANIIQEGFHKSRKVIVVVSRHFIQSRWCIFEYEIAQTWQF
LSSRSGIIFIVLEKVEKSLLRQQVELYRLLSRNTYLEWEDNPLGRHIFW
RRLKNALLDGKASNPEQTAEEEQETATWT.

In one example, the CAR construct comprises an intracellular domain comprising of the costimulatory signalling domains of CD28, CD3ζ and TIR4 (designated entire CAR construct as CD28_{TM}-CD28_{IC}-CD3ζ-TIR4 CAR). In another example, the CD28_{TM}-CD28_{IC}-CD3ζ-TIR4 CAR comprises or consists of the sequence

(SEQ ID NO: 7)
FWVLVWVGGVLACYSLLVTVAFIIFWVRSKRSRLLHSDYMNMTPRRPGP
TRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGE
RRRGKGHDGLYQGLSTATKDTYDALHMQALPPRAGCKKYSRGESIYDAF
VIYSSQNEDWVRNELVKNLEEGVPRFHLCLHYRDFIPGVAIAANIIQEG
FHKSRKVIVVVSRHFIQSRWCIFEYEIAQTWQFLSSRSGIIFIVLEKVE
KSLLRQQVELYRLLSRNTYLEWEDNPLGRHIFWRRLKNALLDGKASNPE
QTAEEEQETATWT.

In one example, the CAR construct comprises the costimulatory signalling domain of CD28 and TIR4 (designated entire CAR construct as CD28_{TM}-CD28_{IC}-TIR4 CAR). In another example, the CD28_{TM}-CD28_{IC}-TIR4 CAR comprises or consists of the sequence

(SEQ ID NO: 8)
FWVLVVVGGVLACYSLLVTVAFIIFWVRSKRSRLLHSDYMNMTPRRPGP
TRKHYQPYAPPRDFAAYRSAGCKKYSRGESIYDAFVIYSSQNEDWVRNE
LVKNLEEGVPRFHLCLHYRDFIPGVAIAANIIQEGFHKSRKVIVVVSRH
FIQSRWCIFEYEIAQTWQFLSSRSGIIFIVLEKVEKSLLRQQVELYRLL
SRNTYLEWEDNPLGRHIFWRRLKNALLDGKASNPEQTAEEEQETATWT.

In one example, the CAR construct comprises the costimulatory signalling domain of TIR4 and CD3ζ. In a further example, the CD28_{TM}-TIR4-CD3ζ CAR comprises or consists of the sequence

(SEQ ID NO: 9)
FWVLVVVGGVLACYSLLVTVAFIIFWVRSAGCKKYSRGESIYDAFVIYS
SQNEDWVRNELVKNLEEGVPRFHLCLHYRDFIPGVAIAANIIQEGFHKS
RKVIVVVSRHFIQSRWCIFEYEIAQTWQFLSSRSGIIFIVLEKVEKSLL
RQQVELYRLLSRNTYLEWEDNPLGRHIFWRRLKNALLDGKASNPEQTAE
EEQETATWTRVKFSRSADAPAYQQGQNQLYNELNLGRREEYDVLDKRRG
RDPEMGGKPRRKNPQEGLYNELQKDKMAEAYSEIGMKGERRRGKGHDGL
YQGLSTATKDTYDALHMQALPPR.

In some examples, the antigen binding domain comprises an antibody or antigen binding fragment thereof.

In some examples, the antibody or antigen binding fragment thereof has binding affinity to a tumour cell antigen. In another examples, the antibody or antigen-binding fragment thereof binds to, or specifically binds to HER2 antigen. In one example, the antigen-binding fragment thereof is an scFv sequence comprising the sequence

(SEQ ID NO: 10)
MDFQVQIFSFLLISASVIMSRQVQLQQSGPELKKPGETVKISCKASGYP
FTNYGMNWVKQAPGQGLKWMGWINTSTGESTFADDFKGRFDFSLETSAN
TAYLQINNLKSEDMATYFCARWEVYHGYVPYWGQGTTVTVSSGGGGSGG
GGSGGGGSDIQLTQSHKFLSTSVGDRVSITCKASQDVYNAVAWYQQKPG
QSPKLLIYSASSRYTGVPSRFTGSGSGPDFTFTISSVQAEDLAVYFCQQ
HFRTPFTFGSGTKLEI.

Without wishing to be bound by theory, the applicant has determined that continuity of the CD28 transmembrane domain (CD28_{TM}) and the CD28 intracellular (CD28_{IC}) costimulatory signalling domain is important for the normal function of CD28. Furthermore, the integrity of the CD28_{TM} and the CD28_{IC} costimulatory signalling domain appear to be important for optimal activation of TIR4. When the CD3ζ domain was added, this further enhanced signal strength. Additionally, the Applicant found that the inclusion of the TIR4 domain at the C-terminal end of the CAR was the most effective position for optimising T cell proliferation and function.

Accordingly, in preferred embodiments, the optimal arrangement of the domains in the expressed protein are:
N-terminus antibody/antigen binding fragment: CD8 hinge CD28_{TM} domain: CD28_{IC} domain: CD3ζ domain: TIR domain C-terminus

In one example, there is provided an anti-HER2-CD28_{TM}-CD28_{IC}-CD3ζ-TIR4 CAR comprising the sequence

(SEQ ID NO: 11)
MDFQVQIFSFLLISASVIMSRQVQLQQSGPELKKPGETVKISCKASGYP
FTNYGMNWVKQAPGQGLKWMGWINTSTGESTFADDFKGRFDFSLETSAN
TAYLQINNLKSEDMATYFCARWEVYHGYVPYWGQGTTVTVSSGGGGSGG
GGSGGGGSDIQLTQSHKFLSTSVGDRVSITCKASQDVYNAVAWYQQKPG
QSPKLLIYSASSRYTGVPSRFTGSGSGPDFTFTISSVQAEDLAVYFCQQ
HFRTPFTFGSGTKLEIEQKLISEEDLNGVTVSSALSNSIMYFSHFVPVF
LPAKPTTTPAPRPPTPAPTIASQPLSLRPEACRPAAGGAVHTRGLDPFW
VLVVVGGVLACYSLLVTVAFIIFWVRSKRSRLLHSDYMNMTPRRPGPTR
KHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERR
RGKGHDGLYQGLSTATKDTYDALHMQALPPRAGCKKYSRGESIYDAFVIE
YSSQNEDWVRNELVKNLEEGVPRFHLCLHYRDFIPGVAIAANIIQEGFH
KSRKVIVVVSRHFIQSRWCIFEYEIAQTWQFLSSRSGIIFIVLEKVEKS
LLRQQVELYRLLSRNTYLEWEDNPLGRHIFWRRLKNALLDGKASNPEQT
AEEQETATWT.

In another example, there is provided an anti-HER2-CD28_{TM}-CD28_{IC}-TIR4 CAR comprising the sequence

(SEQ ID NO: 12.)
MDFQVQIFSFLLISASVIMSRQVQLQQSGPELKKPGETVKISCKASGYP
FTNYGMNWVKQAPGQGLKWMGWINTSTGESTFADDFKGRFDFSLETSAN
TAYLQINNLKSEDMATYFCARWEVYHGYVPYWGQGTTVTVSSGGGGSGG
GGSGGGGSDIQLTQSHKFLSTSVGDRVSITCKASQDVYNAVAWYQQKPG
QSPKLLIYSASSRYTGVPSRFTGSGSGPDFTFTISSVQAEDLAVYFCQQ
HFRTPFTFGSGTKLEIEQKLISEEDLNGVTVSSALSNSIMYFSHFVPVF
LPAKPTTTPAPRPPTPAPTIASQPLSLRPEACRPAAGGAVHTRGLDPFW
VLVVVGGVLACYSLLVTVAFIIFWVRSKRSRLLHSDYMNMTPRRPGPTR
KHYQPYAPPRDFAAYRSAGCKKYSRGESIYDAFVIYSSQNEDWVRNELV
KNLEEGVPRFHLCLHYRDFIPGVAIAANIIQEGFHKSRKVIVVVSRHFI
QSRWCIFEYEIAQTWQFLSSRSGIIFIVLEKVEKSLLRQQVELYRLLSR
NTYLEWEDNPLGRHIFWRRLKNALLDGKASNPEQTAEEEQETATWT

In another example, there is provided an anti-HER2-CD28_{TM}-TIR4-CD3ζ CAR comprising the sequence

(SEQ ID NO: 13)
MDFQVQIFSFLLISASVIMSRQVQLQQSGPELKKPGETVKISCKASGYP
FTNYGMNWVKQAPGQGLKWMGWINTSTGESTFADDFKGRFDFSLETSAN
TAYLQINNLKSEDMATYFCARWEVYHGYVPYWGQGTTVTVSSGGGGSGG
GGSGGGGSDIQLTQSHKFLSTSVGDRVSITCKASQDVYNAVAWYQQKPG
QSPKLLIYSASSRYTGVPSRFTGSGSGPDFTFTISSVQAEDLAVYFCQQ
HFRTPFTFGSGTKLEIEQKLISEEDLNGVTVSSALSNSIMYFSHFVPVF
LPAKPTTTPAPRPPTPAPTIASQPLSLRPEACRPAAGGAVHTRGLDPFW
VLVVVGGVLACYSLLVTVAFIIFWVRSKRSRAGCKKYSRGESIYDAFVI
YSSQNEDWVRNELVKNLEEGVPRFHLCLHYRDFIPGVAIAANIIQEGFH
KSRKVIVVVSRHFIQSRWCIFEYEIAQTWQFLSSRSGIIFIVLEKVEKS
LLRQQVELYRLLSRNTYLEWEDNPLGRHIFWRRLKNALLDGKASNPEQT
AEEEQETATWTRVKFSRSADAPAYQQGQNQLYNELNLGRREEYDVLDKR
RGRDPEMGGKPRRKNPQEGLYNELQKDKMAEAYSEIGMKGERRRGKGHD
GLYQGLSTATKDTYDALHMQALPPR.

In one example, there is provided an anti-HER2-CD28_{TM}-CD28_{IC}-CD3ζ CAR comprising the sequence

(SEQ ID NO: 14)
MDFQVQIFSFLLISASVIMSRQVQLQQSGPELKKPGETVKISCKASGYP
FTNYGMNWVKQAPGQGLKWMGWINTSTGESTFADDFKGRFDFSLETSAN
TAYLQINNLKSEDMATYFCARWEVYHGYVPYWGQGTTVTVSSGGGGSGG
GGSGGGGSDIQLTQSHKFLSTSVGDRVSITCKASQDVYNAVAWYQQKPG
QSPKLLIYSASSRYTGVPSRFTGSGSGPDFTFTISSVQAEDLAVYFCQQ
HFRTPFTFGSGTKLEIEQKLISEEDLNGVTVSSALSNSIMYFSHFVPVF
LPAKPTTTPAPRPPTPAPTIASQPLSLRPEACRPAAGGAVHTRGLDPFW
VLVVVGGVLACYSLLVTVAFIIFWVRSKRSRLLHSDYMNMTPRRPGPTR
KHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERR
RGKGHDGLYQGLSTATKDTYDALHMQALPPR.

In one example, the CAR comprises the sequence of SEQ ID NO:11, SEQ ID NO:12, SEQ ID NO:13 or SEQ ID NO:14, wherein the Myc tag sequence EQKLISEEDL is absent or is replaced with an alternative tag sequence. The skilled person will be familiar with other tags that would be appropriate for use in the constructs of the disclosure. Examples of tags include the hemagglutinin (HA) tag (YPYDVPDYA), 6 His tag (HIS), a FLAG™ tag (DYKDDDDK), the AU1 tag (DTYRYI), the AU5 tag (TDFYLK), GluGlu tag (EYMPME), OLLAS tag (SGFANELGPRLMGK), T7 tag (MASMTGGQQMG), a V5 tag (GKPIPNPLLGLDST), an E-tag (GAPVPYPDPLEPR), and D-Tag (KETAAAKFERQHMDS), and Avi tag (CGLNDIFEAQKIEWHE) or HSV tag (SQPELAPEDPED).

In a fifth aspect, there is provided one or more nucleic acid sequences encoding a CAR comprising the sequence of any one of SEQ ID NOs: 7, 8, 9, 11, 12, 13, 14, 17, 18, 19 or 20.

In one example, the antigen-binding domain is encoded on a first nucleic acid and the transmembrane and intracellular domains are encoded by a second nucleic acid.

In some examples, the nucleic acid sequence encoding the CAR further comprises a signal peptide.

In one example, the dendritic cell is a conventional type 1 (cDC1) cell. In some examples, the dendritic cell is genetically modified.

In one example, the modified dendritic cell is capable of antigen cross-presentation to T cells, an adaptive immune response, or activation of antitumour T cells.

In some examples, the modified dendritic cell is capable of selectively phagocytosing tumour cells (more particularly apoptotic bodies released from tumour cells), cross-presenting a tumour antigen, and/or activating T-cells to respond to the tumour antigen.

In some examples, the modified dendritic cell is capable of cross-presenting tumour antigens (or having tumour antigen cross-presentation), wherein antigen cross-presentation is the ability of a cell to present internalized antigens on type I major histocompatibility complex molecules (MHC I), which is necessary for an efficient cytoxic T cell response against tumour cells.

In a sixth aspect, there is provided a pharmaceutical composition comprising the modified dendritic cell described herein.

In a seventh aspect, there is provided a method of stimulating an adaptive immune response in a subject comprising: administering to the subject a therapeutically effective amount of the modified dendritic cell according to any one of the first to fourth aspects, or the pharmaceutical composition according to the sixth aspect.

In one example, the subject is administered a CAR comprising the sequence of any one of SEQ ID NOs: 7, 8, 9, 11, 12, 13, 17, 18, 19 or 20.

In another example, the subject is administered a modified dendritic cell comprising a CAR having the sequence set forth in any one of SEQ ID NOs: 7, 8, 9, 11, 12, 13, 17, 18, 19 or 20.

In an eighth aspect, there is provided a method of stimulating an adaptive immune response in a subject, comprising administering to the subject a chimeric antigen receptor containing dendritic cell (CAR-DC), the CAR comprising:
(i) an antigen-binding domain;
(ii) a transmembrane domain (TM); and
(iii) an intracellular domain (IC) comprising a toll-interleukin receptor (TIR) intracellular signalling domain and a costimulatory signalling domain selected from a CD3 signalling domain, CD28 signalling domain and a combined CD28 and CD3 signalling domain.

In one example, the subject is administered a CAR comprising the sequence of any one of SEQ ID NOs: 7, 8, 9, 11 12, 13, 17, 18, 19 or 20.

In another example, the subject is administered a modified dendritic cell comprising a CAR having the sequence set forth in any one of SEQ ID NOs: 7, 8, 9, 11, 12, 13, 17, 18, 19 or 20.

In one example according to the seventh or eighth aspects, the subject has cancer. In one example, the cancer is a malignant tumour, solid tumour or liquid tumour. In another example, the method induces phagocytosis of cancer cells in the subject.

In one example, the modified cell or composition induces a tumour-eliminating immune response.

In a ninth aspect, there is provided a method for making a chimeric antigen receptor containing dendritic cell (CAR-DC) population, comprising:
(i) providing a population of cells from a subject;
(ii) culturing the population of cells in a medium comprising an FMS-like tyrosine kinase 3 (Flt3) agonist for at least one day;
(iii) introducing into the population of cells a chimeric antigen receptor (CAR) comprising an antibody or antigen-binding fragment thereof, a CD28 transmembrane domain, and an intracellular domain comprising a toll-interleukin receptor (TIR) intracellular signalling domain and a costimulatory signalling domain selected from a CD3 signalling domain, CD28 signalling domain and a combined CD28 and CD3 signalling domain; and/or
(iv) culturing the cells from (iii) in a medium comprising an FMS-like tyrosine kinase 3 (Flt3) agonist for an amount of time sufficient to form a modified dendritic cell.

In one example, the amount of time sufficient to form the modified dendritic cell is between about 2 days and about 15 days. In another example, the amount of time is about 7 days.

In one example, the population of cells is selected from mononuclear or stem cells from circulation, cord or bone marrow.

In another example, the dendritic cell is a conventional type I dendritic cell (cDC1).

In some examples, introducing the CAR into the dendritic cells comprises introducing a nucleic acid sequence encoding a protein product comprising the antibody or antigen-binding fragment thereof, a CD28 transmembrane domain, and an intracellular domain comprising a toll-interleukin receptor (TIR) intracellular signalling domain and a costimulatory signalling domain selected from a CD3 signalling domain, CD28 signalling domain and a combined CD28 and CD3 signalling domain.

### DESCRIPTION OF THE DRAWINGS

FIG. 1 shows DC-SCRIPT (encoded by the Zfp366 gene) deficiency (resulting in fewer and less functional cDC1s) impaired antigen-specific T cell response within tumour. (A) CD45.2+ WT and Zfp366^{-/-} mice were inoculated with B16-OVA melanoma (5×10⁵) and then adoptively transferred with 2×10⁷ CD45.1+ OT-1 cells/mouse at day 7. Tumour size was measured every 2-3 days (n=5). (B) Enumeration of tumour-infiltrating cDC1s (PI−SiglecHCD11c+MHCII+XCR1+CD11b−), cDC2s (PI−SiglecH−CD11c+MHCII+XCR1−CD11b+) and pDCs (PI−CD11c+MHCII+SiglecH+) from the mice as in A. (C) NK cell number relative to cDC1 cell number. Data shown are the mean±SEM. Each dot represents one mouse. P values calculated using unpaired Student's t-test. *, P<0.05.

FIG. 2 shows cDC1 are required for anti-tumour immunity. (A) WT, Zfp366^{-/-}, CD11c^{cre}Irf8^{+/+} and CD11c^{cre}Irf8^{fl/fl} mice were inoculated with MC38 tumour cells. Tumour size was measured every 2-3 days (n=5). (B) Tumour weight at day 18 following tumour inoculation. (C) Enumeration of tumour infiltrating cDC1s (PI−SiglecH−CD11c+MHC II+XCR1+CD11b−). (D) Tumour size relative to cDC1 cell number. (E) WT, CD11c^{cre}Irf8^{fl/fl}, Rag1^{-/-}, Rag2^{-/-}gc^{-/-}, and Irf8^{fl/fl} ability to suppress tumour growth.

FIG. 3 shows the structure and performance of the CAR constructs absent the TIR signalling domain. (A) shows the construct of the anti-HER2-CD28_{TM}-CD28_{IC}-CD3ζ CAR and empty CAR expressed in retroviral vectors. The anti-HER2-CD28_{TM}-CD28_{IC}-CD3ζ CAR comprises the following left to right: anti-HER2 scFv, myc tag, CD8 hinge, CD28 transmembrane domain (CD28_{TM}), CD28 intracellular domain (CD28_{IC}) and CD3ζ signalling domains, an internal ribosome entry site (IRES) and mCherry label. The empty CAR comprised mCherry only. (B) representative FACS plot of empty vector or anti-HER2-CD28_{TM}-CD28_{IC}-CD3ζ CAR expression in mutuDCs. (C) representative confocal images showing direct GFP (green, Mutu cells) and mCherry (red, retrovirally transduced) fluorescence anti-myc (yellow) and merged with DAPI (blue). The cells were analysed 5 days post-transduction. Scale bar, 100 μm. (D) representative FACS plot of myc expression in empty vector (top plot) or anti-HER2-CD28_{TM}-CD28_{IC}-CD3ζ CAR transduced mutuDCs. (E, F) representative of confocal images showing anti-IRF8 (E, cyan) and anti-DEC205 (F, pink). n=3 independent cell cultures per condition.

FIG. 4 shows CAR+ cDC1s can exhibit direct anti-tumour phagocytic activity. (A) HER2 expression on E0771 tumour cells and mutuDCs transduced with empty vector or anti-HER2-CD28_{TM}-CD28_{IC}-CD3ζ CAR. (B) representative FACS plot of HER2 expression on mutuDCs transduced with empty vector or anti-HER2-CD28_{TM}-CD28_{IC}-CD3ζ CAR before and after coculture with irradiated HER2⁺ E0771 tumours for 2 hours. (C) representative of confocal images showing anti-HER2-CD28_{TM}-CD28_{IC}-CD3ζ CAR transduced mutuDCs (cyan) uptake CTV labelled apoptotic HER2⁺ E0771 tumour cells (red) at 2 hours. (D) representative FACS plot of CTV signal on mutuDCs transduced with empty vector or anti-HER2-CD28_{TM}-CD28_{IC}-CD3ζ CAR before and after coculture with CTV labelled irradiated HER2⁺ or HER2⁻ E0771 tumours for 2 hours. The right bar graph shows the statistics for the flow cytometry data in the left (n=3). (E) representative FACS plot of CTV signal on mutuDCs transduced with empty vector or anti-HER2-CD28_{TM}-CD28_{IC}-CD3ζ CAR before and after coculture with CTV labelled irradiated HER2⁺ E0771 tumours for 15 mins, 30 mins, 45 mins, 1 hour and 2 hours. (F) statistics of E (n=3). Data shown are the mean±SEM. Each dot represents one mouse *P<0.05; **P<0.01; ***P<0.001.

FIG. 5 shows activation of CAR+ cDC1s in response to the tumour cell-associated antigen. (A) empty vector or anti-HER2-CD28_{TM}-CD28_{IC}-CD3ζ CAR transduced mutuDCs were stimulated with LPS, CpG or apoptotic HER2⁺ E0771 tumour cells for 6 hours. The MFI of MHC II was detected by flow cytometry. Bar graph shows the MFI of MHC II for mutuDCs (PI⁻GFP⁺mCherry⁺ MHC II⁺CD11c⁺). (B-C) empty vector or anti-HER2-CD28_{TM}-CD28_{IC}-CD3ζ CAR transduced mutuDCs were stimulated with apoptotic HER2⁺ E0771 tumour cells for 16 hours. The MFI of CD86 (B) and CD80 (C) for mutuDCs (PI⁻GFP⁺mCherry⁺ MHC II⁺CD11c⁺). (D) anti-HER2-CD28_{TM}-CD28_{IC}-CD3ζ CAR transduced mutuDCs (mCherry+) that were cocultured with apoptotic HER2⁺ E0771 tumour cells (achromatic), (E-F) empty vector or anti-HER2-CD28_{TM}-CD28_{IC}-CD3ζ CAR transduced mutuDCs (5×10³) were analysed for antigen cross-presentation to OT-I T cells (2.5×10⁴) in response to the indicated concentrations of OVA-loaded irradiated HER2⁺ E0771 tumour cells. Data are representative of 3 independent experiments. (G) the PI⁺ tumour cells were counted for each condition. Data shown are the mean±SEM. P values calculated using unpaired Student's t-test (A, B and C) or two-way Anova (F and G). *P<0.05; **P<0.01; ***P<0.001; ****P<0.0001.

FIG. 6 shows anti-HER2 CAR+ BM-derived cDC1s exhibit enhanced phagocytic ability in response to tumour specific antigen. (A) WT BM progenitors were transduced with empty vector, intracellular domain truncated anti-HER2 CAR or anti-HER2-CD28_{TM}-CD28_{IC}-CD3ζ CAR bearing retroviruses (all expressing mCherry). The frequency of mCherry-myc- or mCherry⁺myc⁺ pDCs (SiglecH+CD11c+), cDC1 (FVD⁻ SiglecH⁻ MHC II⁺ CD11c⁺ XCR1⁺ CD11b^{low}) and cDC2 (FVD⁻ SiglecH⁻ MHC II⁺ CD11c⁺ XCR1⁻ CD11b⁺) was determined by flow cytometry. (B) histogram shows myc expression in anti-HER2-CD28_{TM}-CD28_{IC}-CD3ζ CAR transduced pDC, cDC2 and cDC1. Myc expression in mCherry⁻ pDC was used as a negative control. (C) histogram shows the expression of indicated cell surface marker in uninfected, or anti-HER2-CD28_{TM}-CD28_{IC}-CD3ζ CAR transduced mCherry⁻myc⁻ or mCherry⁺myc⁺ BM-derived cDC1s (gated as in A). (D) representative FACS plot of CTV signal on BM-derived pDCs, cDC2s and cDC1s (gated as in A) transduced with anti-HER2-CD28_{TM}-CD28_{IC}-CD3ζ CAR before and after coculture with CTV labelled apoptotic HER2⁺ or HER2⁻ E0771 tumours for 2 hours. The CAR expressing cells were marked by mCherry. The right bar graph shows the percentage of CTV⁺ cells within mCherry⁺ or mCherry⁻ populations (n=3). Data shown are the mean±SD. P values calculated using two-way Anova. *P<0.05; **P<0.01.

FIG. 7 shows the predicted protein structures of the various CARs. (A) the structure of TIR4, (B) structure of anti-HER2-CD28_{TM}-CD28_{IC}-CD3 (C) structure of truncated CAR (intra trunc), (D) structure of anti-HER2-CD28_{TM}-CD28_{IC}-TIR4, (E) structure of anti-HER2-CD28_{TM}-TIR4-CD3ζ, (F) structure of anti-HER2-CD28_{TM}-TIR4, and (G) structure of anti-HER2-CD28_{TM}-CD28_{IC}-CD3-TIR4. CAR protein structure was predicted by AlphaFold2. Results are 3D models (bottom) and the predicted aligned error (top). Each amino acid within the protein chain is plotted against itself and against other amino acids. The different function domain were colour-coded and labelled within 3D models (A, B) and the predicted aligned error heatmaps (A-G).

FIG. 8 shows that cDC1s can be activated by TIR4-CAR expression. (A) mutuDCs were transduced with empty vector, anti-HER2 intracellular domain truncated CAR, anti-HER2-CD28_{TM}-TIR4-CD3ζ, anti-HER2-CD28_{TM}-CD28_{IC}-TIR4, anti-HER2-CD28_{TM}-CD28_{IC}-CD3ζ-TIR4 CAR, anti-HER2-CD28_{TM}-TIR4 or anti-HER2-CD28_{TM}-CD28_{IC}-CD3ζ CAR coded retroviruses (all expressing mCherry). The frequency of mCherry+Myc+ mutuDCs was determined by flow cytometry. (B) Representative Confocal images showing the merged image of α-tubulin (cyan) and DAPI (blue). The cells were stained 2 days post-seeding on the slide. Scale bar, 20 μm. (C) MFI of CTV signal on mutuDCs transduced with indicated CAR before and after co-culture with CTV labelled apoptotic HER2⁺ or HER2⁻ E0771 tumours for 2 hours. The bar graph shows the statistics for the FACS data (n=3). (D-E) As indicated in C, the bar graph shows the MFI of HER2 (D) and MHC II (E) in indicated CAR transduced mutuDCs which were co-cultured with CTV labelled apoptotic HER2⁺ or HER2⁻ E0771 tumours for 2 hours relative to the resting cells. (F-H), the bar graph shows the MFI of CD86 (F), CD80 (G), and PD-L1 (H) in indicted CAR transduced mutuDCs which were co-cultured with CTV labelled apoptotic HER2⁺ or HER2⁻ E0771 tumours or stimulated with LPS for 16 hours. Data shown are the mean±SD. P values calculated using two-way Anova. *P<0.05, **P<0.01, ****P<0.0001.

FIG. 9 shows the decreasing autoactivation of the TIR4 containing CAR via modifying the TM domain. (A) mutuDCs were transduced with empty vector, anti-HER2 intracellular domain truncated CAR, anti-HER2-CD28_{TM}-TIR4-CD3ζ, anti-HER2-CD28_{TM}-CD28_{IC}-TIR4, anti-HER2-CD28_{TM}-CD28_{IC}-CD3ζ-TIR4 CAR, anti-HER2-CD28_{TM}-TIR4 anti-HER2-CD28_{TM}-CD28_{IC}-CD3 CAR, anti-HER2-ProCAR1-CD28_{IC}-CD3ζ-TIR4 CAR, anti-HER2-ProCAR2-CD28_{IC}-CD3ζ-TIR4 CAR, anti-HER2-ProCAR3-CD28_{IC}-CD3ζ-TIR4 CAR or anti-HER2-ProCAR4-CD28_{IC}-CD3ζ-TIR4 CAR coded retroviruses. The expression of CD86 in MYC−mCherry− mutuDCs or MYC+mCherry+ mutuDCs in absence of HER2⁺ E0771 tumors was determined by flow cytometry. (B) representative confocal images showing mCherry (red, retrovirally transduced) fluorescence. The cells were analysed 7 days post-transduction. Scale bar, 100 μm. (C-D) empty vector or anti-HER2 intracellular domain truncated CAR or ProCAR1/2/3 or 4-CD28_{IC}-CD3ζ-TIR4 (labelled Pro-CAR1/2/3/4-TIR4) CAR transduced mutuDCs (5×10³) were analysed for antigen cross-presentation to OT-I T cells (2.5×10⁴) in response to the indicated concentrations of OVA expressed apoptotic HER2⁺ E0771 tumour cells. (E-F) The number of GranzymeB⁺ OT-1 and IFNγ⁺ OT-1 in the coculture system as indicated in C were analyzed by flow cytometry. (G) Empty or anti-HER2-ProCAR1-CD28_{IC}-CD3ζ-TIR4 CAR (labelled Pro-CAR2-TIR4 CAR) was transduced into HOXB8 cells then differentiate into cDC1s in presence of OP9-DL1. The Empty or anti-HER2-ProCAR1-CD28_{IC}-CD3ζ-TIR4 CAR (labelled Pro-CAR2-TIR4) expressed cDC1s peritumoral injected into the HER2⁺ E0771 challenged human HER2 transgenic mice at day 5 (purple arrow). Tumour area (length×width) was measured every two to three days.

### KEY TO SEQUENCE LISTING

SEQ ID NO:1: sequence of the CD28_{TM} domain
SEQ ID NO:2: sequence of the CD8 hinge region
SEQ ID NO:3: sequence of the Myc tag
SEQ ID NO:4: sequence of the CD28_{IC} domain
SEQ ID NO:5: sequence of the CD3ζ signalling domain
SEQ ID NO:6: sequence of the TIR4 domain.
SEQ ID NO:7: sequence of CD28_{TM}-CD28_{IC}-CD3ζ-TIR4 CAR lacking CD8 hinge
SEQ ID NO:8: sequence of CD28_{TM}-CD28_{IC}-TIR4 CAR lacking CD8 hinge
SEQ ID NO:9: sequence of CD28_{TM}-TIR4-CD3ζ CAR lacking CD8 hinge
SEQ ID NO:10: sequence of HER2-binding scFv
SEQ ID NO:11: sequence of anti-HER2-CD28_{TM}-CD28_{IC}-CD3ζ-TIR4 CAR
SEQ ID NO:12: sequence of anti-HER2-CD28_{TM}-CD28_{IC}-TIR4CAR
SEQ ID NO:13: sequence of anti-HER2-CD28_{TM}-TIR4-CD3ζ CAR
SEQ ID NO:14: sequence of anti-HER2-CD28-CD3ζ CAR
SEQ ID NO:15: sequence of the truncated CAR
SEQ ID NO:16: sequence of anti-HER2-TIR4 CAR
SEQ ID NO:17: Sequence of anti-HER2-Pro-CAR2-CD28_{IC}-CD3ζ-TIR4 CAR
SEQ ID NO:18: sequence of anti-HER2-Pro-CAR1-CD28_{IC}-CD3ζ-TIR4 CAR
SEQ ID NO:19: sequence of anti-HER2-Pro-CAR3-CD28_{IC}-CD3ζ-TIR4 CAR
SEQ ID NO:20: sequence of anti-HER2-Pro-CAR4-CD28_{IC}-CD3ζ-TIR4 CAR
SEQ ID NO:21: sequence of the HA tag
SEQ ID NO:22: sequence of the HIS tag
SEQ ID NO:23: sequence of the FLAG tag
SEQ ID NO:24: sequence of the AU1 tag
SEQ ID NO:25: sequence of the AU5 tag
SEQ ID NO:26: sequence GluGlu tag
SEQ ID NO: 27: sequence of the OLLAS
SEQ ID NO:28: Sequence of the T7 tag
SEQ ID NO: 29: sequence of the V5 tag
SEQ ID NO: 30: sequence of the E-tag
SEQ ID NO:31: sequence of the D-Tag
SEQ ID NO: 32: sequence of the Avi tag
SEQ ID NO:33: sequence of the HSV tag

### DETAILED DESCRIPTION

### General Techniques

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, recombinant DNA techniques, molecular biology, microbiology, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Definitions

As used herein, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent (%), up or down (higher or lower).

The term “conventional DC” as used herein refers to a dendritic cell which is derived from a common DC progenitor (CDP). Conventional DCs fall into two types, type I and type II. The term typically excludes plasmacytoid DCs. They reside in tissues and, following tissue infection or injury, they become activated and migrate to draining lymph nodes to promote adaptive immune responses.

The term “CAR-DC” as used herein refers to a dendritic cell that expresses a chimeric antigen receptor (CAR).

The term “chimeric antigen receptor (CAR)” as used herein refers to a recombinant fusion protein comprising an extracellular ligand-binding domain (antigen-recognition domain, antigen binding domain), a transmembrane domain and an intracellular signalling transducing domain.

The term “cross-presentation” as used herein refers to the process in which the CAR dendritic cells (CAR-DCs) take up, process, and present antigens (e.g., a tumour cell antigen) on the surface of the cell on a complex with a MHC I molecule. The antigen is then recognized by a T cell.

The term “cross-priming” as used herein refers to the process in which recognition of the antigen by the T cell results in the T cell becoming activated. The activated T cell is then capable of enhanced proliferation, persistence, and/or targeted, enhanced cytotoxicity towards tumour cells expressing that antigen.

The term “allogeneic or heterologous” as used herein refers to a source that is foreign to the host.

The term “autologous” as used herein refers to a source obtained from the same individual.

The term “construct” as used herein is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

The term “composition” as used herein refers to an immunotherapeutic cell population combination with one or more therapeutically acceptable carriers.

The term “therapeutically effective amount” shall be taken to mean a sufficient quantity of an antibody or antigen-binding fragment to reduce or inhibit one or more symptoms of a cellular proliferation disorder to a level that is below that observed and accepted as clinically characteristic of that disorder. The skilled artisan will be aware that such an amount will vary depending on the specific antibody, fragment, and/or particular subject and/or type or severity or level of disease. Accordingly, this term is not to be construed to limit the invention to a specific quantity.

As used herein, the terms “treat,” “treating,” “treatment” and grammatical variations thereof mean subjecting an individual patient to a protocol, regimen, process or remedy, in which it is desired to obtain a physiologic response or outcome in that patient. Since every treated patient may not respond to a particular treatment protocol, regimen, process or remedy, treating does not require that the desired physiologic response or outcome be achieved in each and every patient or patient population. Accordingly, a given patient or patient population may fail to respond or respond inadequately to treatment.

The terms “tumour,” or “cancer” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

### Dendritic Cells

Previously, dendritic cells (DCs) have been characterized as universal ‘all purpose’ antigen-presenting cells, but an important aspect of the control of immune responses is the existence of several different types of DC, each specialized to respond to particular pathogens and to interact with specific subsets of T cells. The term includes DCs that initiate an immune response and/or present an antigen to T lymphocytes and/or provide T-cells with any other activation signal required for stimulation of an adaptive immune response.

As a diverse group of cell types they are widely dispersed throughout the body. They have evolved into a variety of phenotypically and functionally distinct cellular subsets that can be broadly separated into conventional dendritic cells (cDCs), plasmacytoid DCs (pDCs), and monocyte-derived DCs (moDCs). Conventional cDCs can be further divided into type 1 cDC (cDC1s) and type 2 cDCs (cDC2s).

Beyond the expression of CD11c and MHC-II, additional cell surface markers can be used to distinguish DC subsets. cDC1s can be identified as CD141+ DCs. In addition, some cDC1s markers show conserved expression in both mouse and human, such as CLEC9A, CD26, CADM1 and BTLA. In the peripheral lymphoid and non-lymphoid organs such as the lung, gut and LN, cDC1s also can also be identified as CD103+CD11b− cDCs. cDC1s from mouse and human have superior antigen cross-presentation properties which are critical for CD8+ T cell responses to viral or tumour-derived antigens.

The cDC2 subset is defined by the presence of CD11b, Sirpα (CD172a) and CD4 on the cell surface. cDC2s in the peripheral lymphoid and non-lymphoid organs are CD103+CD11b+.

pDCs can be identified by expression of Bst2, B220, SiglecH and express relatively low levels of CD11c and MHC-II. Under inflammatory conditions, moDCs can respond to the proinflammatory chemokines such as CCL2 and CCL7 and upregulate cell surface expression of MHC-II, CD11c and CD11b, thus can be easily confounded as cDC2. Additional markers such as CD64 and MAR-1 can be used to discriminate moDCs from cDC2s.

In addition to their capacity to present antigens directly or indirectly to T cells, DCs produce a plethora of cytokines in response to environmental insults which ultimately fine tune T cells to promote an effective immune response.

Given their natural adjuvants properties, and their unique attributes in promoting T cell priming and recruitment to solid tumours, DCs have long been a focal point of cancer therapies.

The present disclosure is based on the discovery that DCs that have been genetically modified to express a chimeric antigen receptor (CAR), are capable of taking up tumour cells via phagocytosis, micropinocytosis or receptor-mediated endocytosis and promoting cytotoxicity through T-cell cross-priming. The CAR dendritic cells (CAR-DCs) can be used to treat various cancers and malignancies, including solid tumours. Previously described CAR macrophages (CAR-Ms) have not successfully cross-primed T-cells after phagocytosing or pinocytosing tumour cells, and have not been successful in eliminating solid tumours in the clinical trial setting.

The present disclosure describes a method of generating functional CAR-DCs, which selectively engulf tumour cells and cross-present endogenous tumour antigen in a manner that cross-primes tumour antigen-reactive T cells. The CAR-DCs are thus able to generate an adaptive immune response useful for targeting and killing both CAR-Ag+ and CAR-Ag− tumour or cancer cells.

The CAR-DCs can be generated by exposing isolated DC progenitors, such as stem cells (pluripotent, multipotent, hematopoietic, or other stem cells), multipotent progenitors, common myeloid progenitors (CMP), myeloid dendritic cell progenitors (MDP), common dendritic cell progenitor (CDP), bone marrow mononuclear cells, peripheral blood mononuclear cells (PBMC), or splenocytes, to a DC proliferative stimulus such as Flt3L. The cells can then be transduced with the CAR of interest and further exposed to the DC differentiating factor Flt3L for an amount of time sufficient to generate dendritic cell-like cells (DC-like cells) prior to treatment. For example, the cell can be exposed to Flt3L for about 2 to 15 days to promote differentiation.

The present disclosure provides for modified DCs. Numerous studies demonstrate that DCs are limited in the tumour microenvironment and even in cancer patients in general. Further, even if DCs are present, they can induce tolerance or rejection of an antigen, or have no effect at all as they generally have no strong signal instructing them that a tumour cell is foreign or threatening and in need of being eliminated.

A dendritic cell can be a subset of dendritic cells. As an example, a subset of DCs can be, for example, plasmacytoid DC (pDC), a monocyte DC, a myeloid/conventional/classical DC1 (cDC1), or myeloid/conventional/classical DC2 (cDC2).

The present disclosure provides for modified conventional type I dendritic cells (cDC1s), which can be generated by differentiating CAR-DCs. Such cells are effective at antigen cross-priming. Antigen cross-priming refers to the stimulation of antigen-specific naive cytotoxic CD8 T-cells into activated cytotoxic CD8 T-cells by antigen presenting cells that have acquired and cross-presented extracellular antigen, in this case acquired from tumour. Antigen cross presentation refers to the ability of a cell to present internalized antigens on type I major histocompatibility complex molecules (MHC-I). Antigen cross-presentation and cross priming are known to be necessary for an efficient adaptive immune response against tumour cells.

The cDC1s can be identified based on flow cytometry for specific surface protein expression markers as described herein and confirmed by their functional capacity to cross-prime T-cells against engulfed cell-associated antigen. In one example, cDC1s can be sorted based on expression of one or more of NK1.1−, CD19−, TCRb−, SinglecH−, MHC-II+, CD11c+, XCR1−, and CD172a−. In another example in vitro generated cDC1s can be sorted based on expression of one or more of SinglecH−, MHC-II+, CD11c+, XCR1+, and CD172a−.

### Chimeric Antigen Receptors (CARs)

Despite intensive research efforts to define optimal CAR design, a universal CAR structure has not yet been discovered. Several studies indicate that small modifications can have major consequences on the therapeutic outcome.

CARs are designed to comprise an extracellular target-binding domain (e.g., antigen-binding domain, tumour binding domain), a hinge region, a transmembrane domain that anchors the CAR to the cell membrane, and one or more intracellular domains that transmit activation signals. Traditionally CARs use the intracellular signalling motifs derived from T-cell receptors (TCRs) that have been optimised to promote T cell proliferation and function, but these are not as relevant for use in CAR-cDC1 cells which require cDC1 specific effector functions such as tumour cell phagocytosis, antigen processing and presentation, co-stimulatory receptor expression and cytokine secretion. The costimulatory signalling domains, CD28 and 4-1 BB have been most widely used in the CAR-T cell clinical trials, however strikingly different results between these two domains have been seen for the CAR-T cells. Clinical trials for B cell malignancies have shown that CD28-based CAR-T cells are typically undetectable beyond 3 months, whereas 4-11BB-based CAR-T cells can persist in patients for several years after treatment (Fraietta et al, 2018, Nat Med 24, 563-571). It has been suggested that the transmembrane domain of certain costimulatory molecules can be involved in synapse formation or T cell signaling.

The inventors have surprisingly found that in the case of DC cells, the arrangement of the intracellular signalling domains influences the activity of the CAR-DCs. In a preferred arrangement of this invention the linking of the proximal intracellular domain to its corresponding transmembrane domain results in better performance. In the case of the present disclosure the CAR comprises an intracellular domain which is responsible for intracellular signalling following the binding of the extracellular ligand binding domain to the target resulting in the activation of the immune cell and immune response. The signalling (transducing) domain is responsible for the activation of at least one of the normal effector functions of the immune cell in which the CAR is expressed. For example, the effector function of a DC can be an increased survival, differentiation, cytokine secretion, phagocytosis, and/or antigen cross-presentation. Thus, the term “signalling domain” as used herein refers to the portion of a protein which transduces the effector signal function signal and directs the cell to perform a specialized function. Depending on the number of costimulatory signalling domains, CARs can be classified into first (CD3ζ only), second (one costimulatory domain+CD3ζ), or third generation CARs (more than one costimulatory domain+CD3ζ). Costimulatory signalling domains utilized in the present CAR-DCs may similarly be used to increase or decrease the cell's function, persistence, or proliferation. Introduction of CAR molecules into a DC successfully redirects the DC with additional antigen specificity and provides the necessary signals to drive full DC activation and function.

One of the major physiological activation mechanisms for cDC1 is recognition of pathogen-associated molecular patterns (PAMP) via toll-like receptor (TLRs). cDC1s express high levels of TLR3, TLR4, TLR9, TLR11, and TLR13 which recognize different PAMPs often derived from viruses and bacteria. Preferably the intracellular signaling molecule is a motif derived from TLR4. More preferably the intracellular signaling molecule is a toll/interleukin-1 receptor (TIR) from TLR4.

TLR4 agonism has been successfully used in treating colorectal and lung cancer, however other synthetic ligands have also been tested in treating different tumours. For example, TLR7 and TLR8 ligands have been used to treat chronic lymphocytic leukaemia and skin cancer. Similarly, TLR9 ligands have been used for the treatment of lymphoma, renal, skin and brain cancer. Thus, it is expected that different TLRs would initiate different immune responses through variation in cytokine production against different types of cancer.

The TIR domain adopts a flavodoxin-like fold, featuring a central 5 stranded parallel β-sheet that is surrounded by 5 α-helix on both sides of the sheet which are connected by loops. The structure is not dissimilar from the typically used costimulatory signalling domains of CD28_{IC} and/or CD3ζ domains, and thus use in the CAR and maintenance of its flavodoxin-like fold structure may be critical to retain its signalling function.

In response to ligand binding, TLRs will promote a strong proinflammatory response. In cDC1s, the inventors have noted that TLR4 stimulation promotes the MyD88-dependent and TRIF-mediated pathways, leading to their maturation and favours the production of Th1 cytokine via activating NF-kB, AP-1 and IRF transcriptional complexes. TLR4 signalling was also required for DCs to process and cross-present the antigen from dying tumour cells via inducing phagosomal MHC-1 delivery from the endosomal recycling compartment. The choice of this TIR domain in the CAR is an important element to this invention. The presence of TLR signalling is positively correlated with the therapeutic outcome of radiotherapy and chemotherapy.

The differentiation of conventional type 1 dendritic cells (cDC1) from bone marrow progenitors is reliant upon FLT3 (FMS-like tyrosine kinase 3) signaling. Previous studies have demonstrated that incorporating the intracellular domain of FLT3 into the intracellular domain of chimeric antigen receptor (CAR) constructs contributes to the generation of an ample population of cDC1 cells from DC progenitors (as described in WO 2021/127024 A1). However, the cDC1 cells produced through this method exhibit limited capacity to exhibit robust maturation signals upon encountering tumor cells. The selection of a TLR intracellular domain in the present invention overcomes this limitation.

In some examples, the CAR construct domains can be operably linked with a linker. A linker can be any nucleotide sequence capable of linking the domains described herein. For example, the linker can be any amino acid sequence suitable for this purpose (e.g., of a length of 8-80 amino acids, depending on the target-binding domain being used).

The present disclosure provides a modified dentritic cell comprising a chimeric antigen receptor, wherein the chimeric antigen receptor comprises an extracellular domain which specifically binds to a ligand expressed on the surface of a tumour cell, a transmembrane domain, and a signalling domain derived from a toll-like receptor (TLR) combined with another different costimulatory signalling domain, for example to produce a “third generation” CAR comprising two or more signalling domains. In some embodiments, the CAR further comprises another costimulatory signalling domain. In some examples, the other costimulatory signalling domain may be selected from the group consisting of a signalling domain from CD28, 4-1BB (CD137), OX40, CD27, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFI), CD160, CD19, CD4, CD8alpha, CD8beta, IL2R beta, IL2R gamma, IL7R alpha, ITGA6 (VLA6, CD490, CD49a, VLA-1, IGTA4 (CD49D), ITGAD, ITGAM (CD11b), ITGAE (CD103), ITGAL (CD11a), ITGAX (CD11c), ITGB1 (CD29), ITGB2 (CD18), LFA-1 (CD11a), ITGB7, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NCR2, NCR3, NCR1, or NKG2D.

Each costimulatory signalling domain can have unique properties. Differences in the affinity of the scFv, the intensity of antigen expression, the probability of off-tumour toxicity, or the disease to be treated may influence the selection of this domain

Typically, the extracellular antigen-binding domain is linked to the signalling domain of the chimeric antigen receptor (CAR) by a transmembrane domain. The transmembrane domain traverses the cell membrane, anchors the CAR to the DC surface, and connects the extracellular ligand binding domain to the signalling domain, impacting the expression of the CAR on the DC surface. The distinguishing feature of the transmembrane domain in the present disclosure is the ability to be expressed at the surface of a DC to direct an immune cell response against a predefined target cell. The transmembrane domain can be derived from natural or synthetic sources. Alternatively, the transmembrane domain of the present disclosure may be derived from any membrane-bound or transmembrane protein.

In some embodiments, the transmembrane domain may be selected from the group consisting of TNFR2, the alpha, beta or (chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDlIa, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4 (CD49D), IA4, ITGA6 (VLA-6, CD49f, ITGAD (CD11d), ITGAE (CD103), ITGAL (CD11a, LFA-1), ITGAM (CDIIb), ITGAX (CDIIc), ITGB1 (CD29), ITGB2 (CD18), ITGB7, DNAMI (CD226), SLAMF4 (CD244, 2B4), CD84, CD96 (Tactile), CEACAM1, CRT AM, Ly9 (CD229), CD160 (BY55), PSGLI, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, or NKG2C transmembrane domain.

The inventors have surprisingly found that the choice of the transmembrane domain, in some embodiments, is independent of its expression pattern. For example the use of CD28 transmembrane protein works in this invention, despite the fact that CD28 is constitutively expressed predominantly on naïve T cells (roughly 80% of human CD4⁺ T cells and 50% CD8⁺ T cells) and is not currently known to be expressed on DC cells.

In some examples the transmembrane domain is derived from synthetic sources, that is, designed, to modify certain properties of the CAR. In some embodiments the transmembance domain could be one selected from one of the constructs described in WO2021229581, incorporated herein by reference in its entirety.

In some embodiments the synthetic transmembrane domains are designed to enhance properties selected from oliomerisation, specificity (by directing the DC cells response to a predefined target cell) suppression of constitutive signalling or a combination of any these properties.

The transmembrane domain can further comprise a hinge region between the extracellular antigen-binding domain and the transmembrane domain. The term “hinge region” generally means any oligo- or polypeptide that functions to link the transmembrane domain to the extracellular antigen-binding domain. The hinge region may be used to provide more flexibility and accessibility for the extracellular antigen-binding domain. Hinges generally supply stability for efficient CAR expression and activity. The hinge (also in combination with the transmembrane domain), also ensures proper proximity to target. A hinge region may comprise up to 300 amino acids, preferably 5 to 100 amino acids and most preferably 8 to 50 amino acids. Hinge region may be derived from all or parts of naturally-occurring molecules such as CD28, 4-1 BB (CD137), OX-40 (CD134), CD3C, the T cell receptor α or β chain, CD45, CD4, CD5, CD8β, CD8α, CD9, CD16, CD22, CD33, CD37, CD64, CD80, CD86, ICOS, CD154 or from all or parts of an antibody constant region. Alternatively, the hinge region may be a synthetic sequence that corresponds to a naturally-occurring hinge sequence or the hinge region may be an entirely synthetic hinge sequence.

In some examples, the antigen-binding domain is coupled to the transmembrane domain by a hinge region. In some examples, the hinge region comprises a CD8a hinge region. In another example, the hinge region comprises a sequence which is at least 70% or at least 80% or at least 90% or at least 95% identical to SEQ ID NO:2. In another example, the hinge region comprises or consists of the sequence of SEQ ID NO:2.

The CAR can comprise an antibody or antigen binding domain. The antigen binding domain can comprise any domain that binds to an antigen expressed by the targeted cell type (e.g., an antigen expressed by a tumour cell) or a fragment thereof (see e.g., Saar Gill et al. U.S. application Ser. No. 15/747,555 incorporated herein by reference in its entirety). For example, the antigen binding domain can be an antibody (from human, mouse, or other animal), a humanized antibody, a monoclonal antibody, a polyclonal antibody, a synthetic antibody, a camelid antibody, a native receptor or ligand, or a fragment thereof. For example, the antigen binding domain can be a single-chain variable fragment (scFv) of an antibody. The antigen binding domain can be directed to various tumour associated proteins. The tumour antigen may be selected from the group consisting of HER2, EphA2, alphafetoprotein (AFP), carcinoembryonic antigen (CEA), CA-125, MUC-1 antibody, CD19, CD20, CD123, CD22, CD30, SlamF7, CD33, EGFR, BCMA, GD2, CD38, PSMA, B7H3, EPCAM, IL-13Rα2, PSCA, Mesothelin, LewisY, LewisA, CIAX, epithelial tumour antigen (ETA), tyrosinase, melanoma-associated antigen (MAGE), abnormal products of ras or p53, or other proteins found to be more highly enriched on the surface of tumour cells than critical normal tissues. Any protein expressed on the tumour cells can be used in the tumour-related embodiments described herein. Sources of antigen include, but are not limited to, cancer proteins. The antigen can be expressed as an intact protein or portion thereof. The intact protein or a portion thereof can be native or mutagenized. Non-limiting examples of tumour antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-a, Ganglioside G2 (GD2), Ganglioside G3 (GD3), human Epidermal Growth Factor Receptor 2 (FIER-2), human telomerase reverse transcriptase (hTERT), Interleukin-13 receptor subunit alpha-2 (IL-13Ra2), k-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MARTI, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumour-associated glycoprotein 72 (TAG-72), vascular endothelial growth factor R2 (VEGF-R2), and Wilms tumour protein (WT-1), BCMA, NKCS1, EGF1R, EGFR, CD99, CD70, ADGRE2, CCR1, LILRB2, PRAME CCR4, CD5, CD3, TRBC1, TRBC2, TIM-3, Integrin B7, ICAM-1, CD70, CLEC12A and ERBB.

In some examples, the antigen-binding domain comprises an antibody, an antibody fragment, or a receptor ligand. In some embodiments, the antigen-binding domain comprises an scFv, an Fv, a Fab, a ‘Fab’)2, a single domain antibody (SDAB), a VH or VL domain, or a camelid VHH domain.

In one example, the antigen-binding domain is an scFv. scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety. The format of a scFv is generally two variable domains linked by a flexible peptide sequence, either in the orientation VFI-linker-VL or VL-linker-VFI. The orientation of the variable domains within the scFv, depending on the structure of the scFv, may contribute to whether a CAR will be expressed on the DC surface or whether the CAR-DCs target the antigen and signal. In addition, the length and/or composition of the variable domain linker can contribute to the stability or affinity of the scFv. In some examples, the antigen-binding domain comprises a sequence of amino acids which is at least 70% or at least 80% or at least 90% or at least 95% identical to SEQ ID NO:10. In another example, the antigen-binding domain comprises or consists of the sequence of SEQ ID NO:10.

The TLR intracellular domains described herein may be used in CARs which have more than one antigen-binding domain. Thus, in some embodiments, the CAR is a multi-specific CAR comprising two or more antigen-binding domains. The two or more antigen-binding domains may bind to the same or different targets.

### Genetic Modification of DCs

Genetic modification of a DC thereof can be accomplished by transducing a substantially homogeneous cell composition with a recombinant DNA construct. In certain examples, a retroviral vector (either gamma-retroviral or lentiviral) is employed for the introduction of the DNA construct into the cell. For example, a polynucleotide encoding a CAR can be cloned into a retroviral vector and expression can be driven from its endogenous promoter, from the retroviral long terminal repeat, or from a promoter specific for a target cell type of interest. Other viral vectors, or non-viral vectors may be used as well.

For initial genetic modification of a DC thereof to include a CAR, a retroviral vector is generally employed for transduction, however any other suitable viral vector or non-viral delivery system can be used (e.g. lipid nanoparticles). The CAR can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-KB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In other examples, any vector or CAR disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nat. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J Clin. Invest. 89:1817.

The resulting cells can be grown under conditions similar to those for unmodified cells, whereby the modified cells can be expanded and used for a variety of purposes.

Any targeted genome editing methods can be used to place presently disclosed CARs at one or more endogenous gene loci of a presently disclosed immunoresponsive cell. In certain embodiments, a CRISPR system is used to deliver presently disclosed CARs to one or more endogenous gene loci of a presently disclosed immunoresponsive cell.

The disclosure also comprises nucleic acid sequences encoding the CARs described herein, as well as a signal peptide. A signal peptide directs the transport of a secreted or transmembrane protein to the cell membrane and/or cell surface to allow for correct localization of the polypeptide. In particular, the signal peptide of the present disclosure directs the appended polypeptide, i.e. the CAR receptor, to the cell membrane wherein the extracellular antigen-binding domain of the appended polypeptide is displayed on the cell surface, the transmembrane domain of the appended polypeptide spans the cell membrane, and the signalling domain of the appended polypeptide is in the cytoplasmic portion of the cell. In one example, the signal peptide is the signal peptide from human CD8α. In another example, the signal peptide is a fragment of at least 10 amino acids of the CD8α signal peptide that directs the appended polypeptide to the cell membrane and/or cell surface.

The CAR-DCs of the present disclosure may comprise one or more distinct CAR constructs. For example, a dual CAR-DC may be generated by cloning a protein encoding sequence of a first extracellular antigen-binding domain into a viral vector containing one or more costimulatory domains and a signalling domain and cloning a second protein encoding sequence of a second extracellular antigen-binding domain into the same viral vector containing an additional one or more costimulatory domains and a signalling domain resulting in a plasmid from which the two CAR constructs are expressed from the same vector. A tandem CAR-DC, is a DC with a single chimeric antigen polypeptide comprising two distinct extracellular antigen-binding domains capable of interacting with two different cell surface molecules, wherein the extracellular antigen-binding domains are linked together by a flexible linker and share one or more costimulatory domains, wherein the binding of the first or the second extracellular antigen-binding domain will signal through one or more the costimulatory domains and a signalling domain.

### Adaptive T Cell Response

The CAR-DCs described herein are capable of eliciting an adaptive immune response in a subject. An adaptive antitumour T cell response can be initiated or enhanced by antigen cross-presentation or cross-priming from the CAR-DCs. The adaptive antitumour T cell response can comprise, in one example, an increase in T cell function. For example, T cell function can be assessed by the cytotoxic T cell lymphocyte assay (CTL), where an escalating ratio of effector T cells is mixed with target tumour cells for a defined amount of time (generally 4 hours), and tumour cell killing is quantified by tumour luciferase activity. The adaptive antitumour T cell response can also comprise an increase in T cell activation or proliferation. For example, T cell activation or proliferation can be measured by assessing CD4 and CD8 T cell division by FACS analysis for proliferation or for markers of activation, such as cytokine release.

A successful adaptive antitumour T cell response can result in tumour cell cytotoxicity, further tumour cell uptake, and reduction in tumour volume. The antitumour T cell response can directly eliminate antigen positive (Ag+) tumours targeted by the CARs, and indirectly eliminate CAR-Ag− tumour cells (not recognized directly by the CAR), through cross-presentation and epitope spreading. Epitope spreading refers to the broadening of the immune response to include T cell and antibody specificities beyond the antigen that originally triggered the immune response. For example, epitope spreading can result in tumour cells that do not express the antigen targeted by the CAR to be targeted by T cells.

Thus, the present disclosure provides a method of stimulating an adaptive antitumour T cell response in a subject, wherein the method generally comprises administering an effective amount of a CAR-DC described herein to the subject. The CAR-DC targets a tumour or cancer cell, takes up proteins associated with the tumour cells (via phagocytosis, macropinocytosis or receptor-mediated endocytosis). the tumour or cancer cell and cross-presents tumour antigens to the subject's T cells. Accordingly, the CAR-DCs directly target antigen positive (Ag+) tumour or cancer cells for elimination and/or indirectly targets CAR-antigen negative (Ag−) tumour or cancer cells for elimination through cross-presentation and epitope spreading.

### Compositions

The disclosure also provides pharmaceutical compositions. In one example, the pharmaceutical compositions comprises a plurality of CAR-DCs together with a pharmaceutically acceptable excipient. The pharmaceutically acceptable excipient may be a diluent, a binder, a filler, a buffering agent, a pH modifying agent, a disintegrant, a dispersant, a preservative, a lubricant, taste-masking agent, a flavoring agent, or a colouring agent.

Compositions comprising the presently disclosed CAR-DCs can be provided as sterile liquid preparations, e.g., isotonic aqueous solutions, suspensions, emulsions, dispersions, or viscous compositions, which may be buffered to a selected pH. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the CAR-DCs in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. Additional auxiliary substances may be included such as emulsifying agents, wetting agents, dispersing agents, pH buffering agents, gelling agents, preservatives or colours. Various additives which enhance the stability and sterility of the compositions, including antimicrobial preservatives, antioxidants, chelating agents, and buffers, can be added.

The quantity of cells to be administered will vary for the subject being treated. In a one example, between about 10³ and about 10¹⁰, between about 10⁵ and about 10⁹, or between about 10⁶ and about 10⁸ of the presently disclosed CAR-DCs are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed CAR-DCs are administered to a human subject. In certain embodiments, between about 1×10⁷ and 5×10⁸ of the presently disclosed CAR-DCs are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The person skilled in the art can readily determine the amount of cells and optional additives, vehicles, and/or carrier in compositions and to be administered in the methods described herein. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

The compositions described herein may be provided systemically or parenterally to a subject for inducing and/or enhancing an immune response to an antigen and/or treating and/or preventing a neoplasm, pathogen infection, or infectious disease.

In certain examples, the presently disclosed CAR-DCs or compositions comprising thereof are directly injected into a tumour or organ of interest (e.g., an organ affected by a neoplasia). Alternatively, the presently disclosed CAR-DCs or compositions comprising thereof are provided indirectly to the organ of interest, for example, by administration into the circulatory system (e.g., the tumour vasculature). Expansion and differentiation agents can be provided prior to, during or after administration of the cells or compositions to increase production of T cells, NK cells, or CTL cells in vitro or in vivo.

The presently disclosed CAR-DCs can be administered in any physiologically acceptable vehicle, normally intravascularly, although they may also be introduced into bone or other convenient site where the cells may find an appropriate site for regeneration and differentiation (e.g., lymphatics). The presently disclosed CAR-DCs can comprise a purified population of cells. Those skilled in the art can readily determine the percentage of the presently CAR-DCs in a population using various well-known methods, such as fluorescence activated cell sorting (FACS). Suitable ranges of purity in populations comprising the presently disclosed CAR-DCs are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

Administration of the composition can be autologous or heterologous. For example, CAR-DCs, or progenitors can be derived or obtained from one subject, and administered to the same subject or a different, compatible subject.

### Treatment

The disclosure also provides methods for treating cancer in a subject or reducing or preventing cancer recurrence in a subject comprising administration of an effective amount of the modified dendritic cell or a CAR-DC described herein which target an antigen expressed by the cancer cell. In one example, the subject is administered the modified dendritic cell according to any one of the first to fourth aspects. In one example, the subject is administered the pharmaceutical composition according to the sixth aspect. In one example, the subject is administered a CAR comprising the sequence of any one of SEQ ID NOs: 7, 8, 9, 11, 12 or 13, 17, 18, 19.

A recurrence occurs when the cancer comes back after the initial treatment. This can happen weeks, months, or even years after the primary or original cancer was treated.

The methods described herein are particularly efficacious for treating a semi-solid or solid tumour. Traditional chimeric antigen receptor (CAR) T cells exhibit only a 1% complete response in solid tumours in clinical trials thus far. Solid tumours escape CAR T recognition if not all cells express the target antigen. Accordingly, successfully creating an adaptive immune response in patients would overcome the failures of both types of immunotherapy.

In one example, the cancer is selected from the group consisting of lung, breast, colorectal, prostate, cervical, ovarian, skin, melanoma, stomach, pancreatic, liver, brain, glioblastoma, neuroblastoma, throat, esophageal, bladder, and head and neck cancer.

In one example, the cancer is a haematological malignancy. Hematologic malignancies include leukemias, lymphomas, multiple myeloma, and subtypes thereof. Lymphomas can be classified by various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art. Plasma cell malignancies include lymphoplasmacytic lymphoma, plasmacytoma, and multiple myeloma.

Administration to a subject of CAR-DCs described herein may be by one or more of subcutaneously, intradermally, intratumourally, intranodally, intramedullary, intramuscularly, by intravenous or intralymphatic injection, or intraperitoneally. In one example, the administration is intra-tumourally.

In a further aspect, the disclosure also provides use of a chimeric antigen receptor containing dendritic cell (CAR-DC) comprising:
(i) an antigen-binding domain;
(ii) a transmembrane domain (TM); and
(iii) an intracellular domain (IC) comprising a toll-interleukin receptor (TIR) intracellular signalling domain and a costimulatory signalling domain selected from a CD3 signalling domain, CD28 signalling domain and a combined CD28 and CD3 signalling domain;
in the manufacture of a medicament for stimulating an adaptive immune response in a subject.

In one example, the medicament can be used to treat a semi-solid or solid tumour or haematological malignancy as described herein.

Cancers amenable to treatment according to the methods of the disclosure include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumours (cerebellar astrocytoma, cerebral astrocytoma/malignant glioma, ependymoma, medulloblastoma, supratentorial primitive neuroectodermal tumours, visual pathway and hypothalamic gliomas), breast cancer, bronchial adenomas/carcinoids, Burkitt lymphoma, carcinoid tumours (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumour, endometrial cancer, ependymoma, esophageal cancer, Ewing's sarcoma in the Ewing family of tumours, extracranial germ cell tumour (childhood), extragonadal germ cell tumour, extrahepatic bile duct cancer, eye cancers (intraocular melanoma, retinoblastoma), gallbladder cancer, gastric (stomach) cancer, gastrointestinal carcinoid tumour, gastrointestinal stromal tumour, germ cell tumours (childhood extracranial, extragonadal, ovarian), gestational trophoblastic tumour, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumour), ovarian germ cell tumour, ovarian low malignant potential tumour, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumours (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumours, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumour (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumour (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenstrom macroglobulinemia, or Wilms tumour (childhood).

Methods described herein are generally performed on a subject in need thereof. A subject in need of the therapeutic methods described herein can be a subject having, diagnosed with, suspected of having, or at risk for developing a cancer or proliferative disease, disorder, or condition. A determination of the need for treatment will typically be assessed by a history, physical exam, or diagnostic tests consistent with the disease or condition at issue.

Administration of CAR-DC therapy as described herein may be a single event or over a time course of treatment. For example, DC-based therapy can be administered daily, weekly, bi-weekly, or monthly. For more chronic conditions, treatment could extend from several weeks to several months or years.

In some examples, CAR-DC therapy may be administered concurrent with, before or after conventional cancer treatment modalities. In one example, CAR-DC therapy can be administered simultaneously or sequentially with another agent, such as an anti-cancer agent. In some examples the anti-cancer agent is a chemotherapy agent, a radiotherapy agent or immunotherapy agent.

The CAR-DCs described herein may be administered in combination with agents that inhibit immunosuppressive pathways, including but not limited to, inhibitors of TGFβ, interleukin 10 (IL-10), adenosine, VEGF, indoleamine 2,3 dioxygenase 1 (ID01), indoleamine 2,3-dioxygenase 2 (ID02), tryptophan 2-3-dioxygenase (TDO), lactate, hypoxia, arginase, and prostaglandin E2. In another example, the CAR-DCs or a population of CAR-DCs of the present disclosure may be used in combination with T-cell checkpoint inhibitors, including but not limited to, anti-CTLA4 (such as Ipilimumab) anti-PD1 (such as Pembrolizumab, Nivolumab, Cemiplimab), anti-PDL1 (such as Atezolizumab, Avelumab, Durvalumab), anti-PDL2, anti-BTLA, anti-LAG3, anti-TIM3, anti-VISTA, anti-TIGIT, and anti-KIR.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive. The present disclosure includes the following non-limiting examples.

### EXAMPLES

It will be appreciated by persons skilled in the art that numerous variations and/or modifications may be made to the above-described embodiments and the following examples, without departing from the broad general scope of the present disclosure. The present embodiments are, therefore, to be considered in all respects as illustrative and not restrictive. Those skilled in the art should, in light of the present disclosure, appreciate that many changes can be made to specific embodiments which are disclosed and still obtain a similar result without departing from the spirit and scope of the invention.

### Materials and Methods

### Mice

All experiments were performed using wild type (WT) C57BL/6 mice or C57BL/6 CD45.2 Zfp366^{+/+} or CD45.2 Zfp366^{-/-} mice maintained under specific pathogen-free conditions. For experiments, mice between the age of 8-12 weeks were used and all experiments subject to ethics committee approval.

### Production of Retroviral Particles

Retroviral particles were produced by transient transfection of three plasmids into 293T cells. 3.5×10⁶ 293T cells were seeded in 10 cm dishes in 10 ml of DMEM with 10% FCS. One day later the DMEM media was replaced with prewarmed OptiMEM media with 10% FCS. For each dish, a plasmid mix was prepared containing the packaging Gag/pol plasmid (1.2 μg), the envelope plasmid (VSV-G, 0.6 mg) and the transfer retroviral plasmid (3.6 mg) then added to 100 μl serum-free OptiMEM media. 30 ml FuGENE-6 reagent was added in this system and incubate at room temperature for 15 min. The plasmid/FuGEENE mix was added to the 293T cells and dishes incubated at 37° C., 5% CO₂ overnight. Media was changed the next day with prewarmed DMEM with 10% FCS. Virus containing supernatant was harvest after 48 hours for transduction and used immediately or stored at −80° C.

### Cell Transduction

The 6 well plate was coated with RetroNectin (Takara) at 4° C. for overnight. Retroviral supernatants were centrifuged onto the coated plate for 2 hours at 2000 G at 32° C. The cells were then and centrifuged at 1700 rpm for 20 min at 32° C. The transduction efficiency was checked via flow cytometry after 48 hours.

### Cell Lines and Primary Cell Culture

MutuDCs originated from spleen tumours in CD11c:SV40LgT-transgenic C57BL/6 mice. The cells were cultured in IMDM with 10% FCS, and 50 mM b-mercaptoethanol. Parental and human HER2 transduced E0771 cells were cultured in DMEM with 10% FCS.

### Immunofluorescence Analysis

CAR transduced cDC1s were seeded on 8-well glass chamber slides (ibdi) at a concentration of 1×10³ cells/well two days before immunofluorescence stain. The cells were washed, fixed, and blocked, then incubate with anti-tubulin-Alexa 647 or anti-MYC APC overnight at cold room. After staining, the slides were washed then fixed with ProLong Gold antifade mounting medium with DAPI (Invitrogen) for confocal microscopy, imaged by confocal microscopy (Zeiss LSM 780), and then processed in ImageJ.

### Heat Shock and Irradiation Induced Tumour Cell Apoptosis

Cell lines (HER2⁺ or HER2⁻ E0771) were cultured in 10 cm dishes until 80% confluence. Cells were then removed via trypsin-EDTA solution and resuspended in 1 ml of DMEM and subjected to heat shock (44° C. for 50 min) or irradiation (3000 rad). To labelled tumour cells with CTV, cells were washed and resuspend in 5 nM of CTV for 15 min in 37° C. after heat shock.

### In Vitro Phagocytosis Assay

Apoptotic HER2⁺ or HER2⁻ E0771 tumour cells were labelled with CTV then cocultured with CAR transduced BMDCs or mutuDCs at 1:1 ratio for the indicated times in U bottom 96 well plate. Following coculture, the plate was placed on ice immediately, and stained with antibody cocktails for FACS analysis.

### In Vitro Generation of DCs

Hips, femurs, and tibias from mice were crushed into FACS buffer (PBS+0.5% BSA, Sigma-Aldrich) and erythrocytes were removed with ACK lysis buffer. BM cells were cultured at 1.5×10⁶ cells/ml in RPMI-1640 supplemented with 10% heat-inactivated fetal calf serum, 2 mM L-Glutamine (GIBCO), 50 μM 2-mercaptoethanol (Sigma-Aldrich) 100 U/ml penicillin/streptomycin (GIBCO) containing FLT3L (200 ng/ml, BioXCell) for 7 days to generate conventional dendritic cells (cDCs) and plasmacytoid (pDCs).

### Flow Cytometry

Single-cell suspensions of bone marrow were resuspended in FACS buffer and stained at 4° C. All analyses were performed on a BD FACSCanto or BD LSRFortessa (BD Biosciences).

### Cell Sorting and Magnetic Enrichment

Harvested spleens were digested by collagenase (Worthington) and DNase I (Sigma). Erythrocytes were removed with ACK lysis buffer. Light density cells were collected by a density centrifugation procedure. CD11c⁺ DCs were enriched by CD11c magnetic beads (Miltenyi). The purity of the enriched CD11c⁺ DC preparation was >90%. cDC1s were sorted as PI⁻NK1.1⁻CD19⁻TCRb⁻SiglecH⁻MHC-II⁺CD11c⁺XCR1⁺CD172a⁻. cDC2s were sorted as PI⁻NK1.1⁻CD19⁻TCRb⁻SiglecH⁻MHC-II⁺CD11c⁺XCR1⁻CD172a⁺. The in vitro generated DCs were sorted as PI⁻SiglecH⁻MHC-II⁺CD11c⁺XCR1⁺CD172a⁻ (cDC1) and PI⁻SiglecH⁻MHC-II⁺CD11c⁺XCR1⁻CD172a⁺ (cDC2). For OVA-specific OT-I or OT-II cells enrichment, lymph node (LN) was collected and passed through a 70 mm sieve. LN cells were negatively selected with a lineage (Lin) cocktail including mAbs against MHC-II, B220, MAC-1, Ly6G, CD44 and TER119, and positively selected with CD4 (for OT-II enrichment) or CD8 (for OT-1 enrichment), using with BioMag Goat Anti-Rat IgG beads (Qiagen). Cells were checked for purity (>99%) by analytical flow cytometry using CD8 or CD4, TCRva2 antibody.

### Antigen Presentation and Cross-Presentation Assays

For in vitro antigen presentation assay, purified OVA-specific SIINFEKL-specific CD8⁺ (OT-I T cells) were labelled with cell Trace™ violet (CTV) according to manufacturer's recommendations. 5×10⁴ OT-I T cells were co-cultured with purified CAR transduced cDC1s with or without OVA-loaded HER2⁺ E0771 tumour cells which were irradiated (3000 rads). 60-72 h post stimulation, numbers of proliferating T cells were measured by flow cytometry.

### Design and Construction of Retroviral Vectors

The coding segment of the anti-HER2 scFv, CD8 hinge, CD28_{TM} and mCherry were obtained by PCR from anti-HER2-CD28_{TM}-CD28_{IC}-CD3ζ-mCherry CAR plasmid in pMSCV backbone using the following primers containing 20 bp overlap with the insert fragment.

Forward primer:
GTCTGTAGCGACCCTTTGCAGGCAGCGG
Reverse primer:
CACCCAAAAGGGATCCAGCCCCCTCGTG

The designed DNA insert fragment coding for intracellular signalling tails were ordered from IDT (Integrated DNA technology). It contains 20 bp overlaps at the 3′-end and 5′-end with the backbone vector.

The PCR product was then assembled using Gibson clone (NEB). The correct clone which contains the designed plasmid was selected after Sanger sequencing.

Forward primer:
GGCTGGATCCCTTTTGGGTG.
Reverse primer:
ACAGACCTTGCATTCCTTTGGC

### Cloned Sequences

The sequences used herein are provided in Table 1 below.

TABLE 1
Sequences of possible CAR domains.

| Domain | Amino acid sequence |
|---|---|
| HER2 scFv | MDFQVQIFSFLLISASVIMSRQVQLQQSGPELKKPGETVKISCKASGYPFTNYGMN |
| | WVKQAPGQGLKWMGWINTSTGESTFADDFKGRFDFSLETSANTAYLQINNLKSE |
| | DMATYFCARWEVYHGYVPYWGQGTTVTVSSGGGGSGGGGSGGGGSDIQLTQS |
| | HKFLSTSVGDRVSITCKASQDVYNAVAWYQQKPGQSPKLLIYSASSRYTGVPSRF |
| | TGSGSGPDFTFTISSVQAEDLAVYFCQQHFRTPFTFGSGTKLEI (SEQ ID NO: 10) |
| CD8 hinge | NGVTVSSALSNSIMYFSHFVPVFLPAKPTTTPAPRPPTPAPTIASQPLSLRPEACR |
| | PAAGGAVHTRGLDP (SEQ ID NO: 2) |
| Myc tag | EQKLISEEDL (SEQ ID NO: 3) |
| HA tag | YPYDVPDYA (SEQ ID NO: 21) |
| HIS tag | HHHHHH (SEQ ID NO: 22) |
| FLAG tag | DYKDDDDK (SEQ ID NO: 23) |
| AU1 Tag | DTYRYI (SEQ ID NO: 24) |
| AU5 Tag | TDFYLK(SEQ ID NO: 25) |
| GluGlu tag | EYMPME (SEQ ID NO: 26) |
| OLLAS tag | SGFANELGPR LMGK (SEQ ID NO: 27) |
| T7 tag | MASMTGGQQMG (SEQ ID NO: 28) |
| V5 | GKPIPNPLLGLDST (SEQ ID NO: 29) |
| E-Tag | GAPVPYPDPLEPR (SEQ ID NO: 30) |
| D-Tag | KETAAAKFERQHMDS (SEQ ID NO: 31) |
| Avi Tag | CGLNDIFEAQKIEWHE (SEQ ID NO: 32) |
| HSV Tag | SQPELAPEDPED (SEQ ID NO: 33) |
| CD28_{TM} | FWVLVVVGGVLACYSLLVTVAFIIFWVRS (SEQ ID NO: 1) |
| CD28_{IC} | KRSRLLHSDYMNMTPRRPGPTRKHYQPYAPPRDFAAYRS (SEQ ID NO: 4) |
| CD3ζ | RVKFSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDALHM |
| | QALPPR (SEQ ID NO: 5) |
| TIR4 | AGCKKYSRGESIYDAFVIYSSQNEDWVRNELVKNLEEGVPRFHLCLHYRDFIPGV |
| | AIAANIIQEGFHKSRKVIVVVSRHFIQSRWCIFEYEIAQTWQFLSSRSGIIFIVLEKVE |
| | KSLLRQQVELYRLLSRNTYLEWEDNPLGRHIFWRRLKNALLDGKASNPEQTAEEE |
| | QETATWT (SEQ ID NO: 6) |
| ProCAR2_{TM} | FWLTVALILGIFLGTFIAFWVVYLLWVRS (SEQ ID NO: 17) |
| ProCAR1_{TM} | FWLVLILLTFVLFVFILYWVITWYLIWVRS (SEQ ID NO: 18) |
| ProCAR3_{TM} | FWLLFILVAILGGLFGAIVAFLLALWVRS (SEQ ID NO: 19) |
| ProCAR4_{TM} | FWLLVALLALLAVIAALLAAIFALWVRS (SEQ ID NO: 20) |
| CD28_{TM}- | FWVLVVVGGVLACYSLLVTVAFIIFWVRSKRSRLLHSDYMNMTPRRPGPTRKHYQ |
| CD28_{IC}- | PYAPPRDFAAYRSRVKFSRSADAPAYQQGQNQLYNELNLGRREEYDVLDKRRG |
| CD3ζ-TIR4 | RDPEMGGKPRRKNPQEGLYNELQKDKMAEAYSEIGMKGERRRGKGHDGLYQGL |
| | STATKDTYDALHMQALPPRAGCKKYSRGESIYDAFVIYSSQNEDWVRNELVKNLE |
| | EGVPRFHLCLHYRDFIPGVAIAANIIQEGFHKSRKVIVVVSRHFIQSRWCIFEYEIAQ |
| | TWQFLSSRSGIIFIVLEKVEKSLLRQQVELYRLLSRNTYLEWEDNPLGRHIFWRRL |
| | KNALLDGKASNPEQTAEEEQETATWT (SEQ ID NO: 7). |
| CD28_{TM}- | FWVLVVVGGVLACYSLLVTVAFIIFWVRSKRSRLLHSDYMNMTPRRPGPTRKHYQ |
| CD28_{IC}-TIR4 | PYAPPRDFAAYRSAGCKKYSRGESIYDAFVIYSSQNEDWVRNELVKNLEEGVPRF |
| | HLCLHYRDFIPGVAIAANIIQEGFHKSRKVIVVVSRHFIQSRWCIFEYEIAQTWQFLS |
| | SRSGIIFIVLEKVEKSLLRQQVELYRLLSRNTYLEWEDNPLGRHIFWRRLKNALLDG |
| | KASNPEQTAEEEQETATWT (SEQ ID NO: 8). |
| CD28_{TM}- | FWVLVVVGGVLACYSLLVTVAFIIFWVRSAGCKKYSRGESIYDAFVIYSSQNEDWV |
| TIR4-CD3ζ | RNELVKNLEEGVPRFHLCLHYRDFIPGVAIAANIIQEGFHKSRKVIVVVSRHFIQSR |
| | WCIFEYEIAQTWQFLSSRSGIIFIVLEKVEKSLLRQQVELYRLLSRNTYLEWEDNPL |
| | GRHIFWRRLKNALLDGKASNPEQTAEEEQETATWTRVKFSRSADAPAYQQGQN |
| | QLYNELNLGRREEYDVLDKRRGRDPEMGGKPRRKNPQEGLYNELQKDKMAEAY |
| | SEIGMKGERRRGKGHDGLYQGLSTATKDTYDALHMQALPPR (SEQ ID NO: 9). |

As controls, the inventors made a truncated CAR which comprised of an Anti-HER2 scFv, Myc tag, 008 hinge, CD28_{TM} and a truncated CD28_{IC} domain which consisted of the sequence KRSR.

The sequences of the exemplified CARs are shown below:

The full amino acid sequence of the anti-HER2-CD28_{TM}-CD28_{IC}-CD33 CAR is as follows:
(SEQ ID NO: 14)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEACRPAAGGAVHTRGLDPFWVLVVVGGVLACYSLLVTVAFIIFWVRSKRSRLLHS
DYMNMTPRRPGPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGERRRGKGHDGL
YQGLSTATKDTYDALHMQALPPR.
The full amino acid sequence of the anti-HER2-CD28_{TM}-CD28_{IC}-TIR4 CAR is as follows:
(SEQ ID NO: 12)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEACRPAAGGAVHTRGLDPFWVLVVVGGVLACYSLLVTVAFIIFWVRSKRSRLLHS
DYMNMTPRRPGPTRKHYQPYAPPRDFAAYRSAGCKKYSRGESIYDAFVIYSSQNEDWVRNE
LVKNLEEGVPRFHLCLHYRDFIPGVAIAANIIQEGFHKSRKVIVVVSRHFIQSRWCIFEYEIAQTW
QFLSSRSGIIFIVLEKVEKSLLRQQVELYRLLSRNTYLEWEDNPLGRHIFWRRLKNALLDGKASN
PEQTAEEEQETATWT.
The full amino acid sequence of the anti-HER2-CD28_{TM}-TIR4-CD3ζ CAR is as follows:
(SEQ ID NO: 13)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEACRPAAGGAVHTRGLDPFWVLVVVGGVLACYSLLVTVAFIIFWVRSKRSRAGCK
KYSRGESIYDAFVIYSSQNEDWVRNELVKNLEEGVPRFHLCLHYRDFIPGVAIAANIIQEGFHKS
RKVIVVVSRHFIQSRWCIFEYEIAQTWQFLSSRSGIIFIVLEKVEKSLLRQQVELYRLLSRNTYLE
WEDNPLGRHIFWRRLKNALLDGKASNPEQTAEEEQETATWTRVKFSRSADAPAYQQGQNQL
YNELNLGRREEYDVLDKRRGRDPEMGGKPRRKNPQEGLYNELQKDKMAEAYSEIGMKGERR
RGKGHDGLYQGLSTATKDTYDALHMQALPPR.
The full amino acid sequence of the anti-HER2-CD28_{TM}-TIR4 CAR is as follows:
(SEQ ID NO: 16)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEACRPAAGGAVHTRGLDPFWVLVVVGGVLACYSLLVTVAFIIFWVRSKRSRKKYS
RGESIYDAFVIYSSQNEDWVRNELVKNLEEGVPRFHLCLHYRDFIPGVAIAANIIQEGFHKSRKV
IVVVSRHFIQSRWCIFEYEIAQTWQFLSSRSGIIFIVLEKVEKSLLRQQVELYRLLSRNTYLEWED
NPLGRHIFWRRLKNALLDGKASNPEQTAEEEQETATWT.
The full amino acid sequence of the anti-HER2-CD28_{TM}-CD28_{IC}-CD3ζ-TIR4 CAR is as follows:
(SEQ ID NO: 11)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEACRPAAGGAVHTRGLDPFWVLVVVGGVLACYSLLVTVAFIIFWVRSKRSRLLHS
DYMNMTPRRPGPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGERRRGKGHDGL
YQGLSTATKDTYDALHMQALPPRAGCKKYSRGESIYDAFVIYSSQNEDWVRNELVKNLEEGV
PRFHLCLHYRDFIPGVAIAANIIQEGFHKSRKVIVVVSRHFIQSRWCIFEYEIAQTWQFLSSRSGII
FIVLEKVEKSLLRQQVELYRLLSRNTYLEWEDNPLGRHIFWRRLKNALLDGKASNPEQTAEEE
QETATWT.
The full amino acid sequence of the intracellular truncated CAR is as follows:
(SEQ ID NO: 15)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEACRPAAGGAVHTRGLDPFWVLVVVGGVLACYSLLVTVAFIIFWVRSKRSR.
The full amino acid sequence of the anti-HER2-Pro-CAR2-CD28_{IC}-CD3ζ-TIR4 CAR is as
follows:
(SEQ ID NO: 17)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEAARPAAGGAVHTRGLDPFWLTVALILGIFLGTFIAFWVVYLLWVRSKRSRLLHSD
YMNMTPRRPGPTRKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGERRRGKGHDGLYQ
GLSTATKDTYDALHMQALPPRAGCKKYSRGESIYDAFVIYSSQNEDWVRNELVKNLEEGVPRF
HLCLHYRDFIPGVAIAANIIQEGFHKSRKVIVVVSRHFIQSRWCIFEYEIAQTWQFLSSRSGIIFIV
LEKVEKSLLRQQVELYRLLSRNTYLEWEDNPLGRHIFWRRLKNALLDGKASNPEQTAEEEQET
ATWT.
The full amino acid sequence of the anti-HER2-Pro-CAR1-CD28_{IC}-CD3ζ-TIR4 CAR is as
follows:
(SEQ ID NO: 18)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEAARPAAGGAVHTRGLDPFWLVLILLTFVLFVFILYWVITWYLIWVRSKRSRLLHSD
YMNMTPRRPGPTRKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGERRRGKGHDGLYQ
GLSTATKDTYDALHMQALPPRAGCKKYSRGESIYDAFVIYSSQNEDWVRNELVKNLEEGVPRF
HLCLHYRDFIPGVAIAANIIQEGFHKSRKVIVVVSRHFIQSRWCIFEYEIAQTWQFLSSRSGIIFIVL
EKVEKSLLRQQVELYRLLSRNTYLEWEDNPLGRHIFWRRLKNALLDGKASNPEQTAEEEQETA
TWT.
The full amino acid sequence of the anti-HER2-Pro-CAR3-CD28_{IC}-CD3ζ-TIR4 CAR is as
follows:
(SEQ ID NO: 19)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEAARPAAGGAVHTRGLDPFWLLFILVAILGGLFGAIVAFLLALWVRSKRSRLLHSDY
MNMTPRRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGHDGLYQG
LSTATKDTYDALHMQALPPRAGCKKYSRGESIYDAFVIYSSQNEDWVRNELVKNLEEGVPRFH
LCLHYRDFIPGVAIAANIIQEGFHKSRKVIVVVSRHFIQSRWCIFEYEIAQTWQFLSSRSGIIFIVLE
KVEKSLLRQQVELYRLLSRNTYLEWEDNPLGRHIFWRRLKNALLDGKASNPEQTAEEEQETAT
WT.
The full amino acid sequence of the anti-HER2-Pro-CAR4-CD28_{IC}-CD3ζ-TIR4 CAR is as
follows:
(SEQ ID NO: 20)
MDFQVQIFSFLLISASVIMSRQVQLQQSGPELKKPGETVKISCKASGYPFTNYGMNWVKQAPG
QGLKWMGWINTSTGESTFADDFKGRFDFSLETSANTAYLQINNLKSEDMATYFCARWEVYHG
YVPYWGQGTTVTVSSGGGGSGGGGSGGGGSDIQLTQSHKFLSTSVGDRVSITCKASQDVYN
AVAWYQQKPGQSPKLLIYSASSRYTGVPSRFTGSGSGPDFTFTISSVQAEDLAVYFCQQHFRT
PFTFGSGTKLEIEQKLISEEDLNGVTVSSALSNSIMYFSHFVPVFLPAKPTTTPAPRPPTPAPTIA
SQPLSLRPEAARPAAGGAVHTRGLDPFWLLVALLALLAVIAALLAAIFALWVRSKRSRLLHSDYM
NMTPRRPGPTRKHYQPYAPPRDFAAYRSRVKFSRSADAPAYQQGQNQLYNELNLGRREEYD
VLDKRRGRDPEMGGKPRRKNPQEGLYNELQKDKMAEAYSEIGMKGERRRGKGHDGLYQGL
STATKDTYDALHMQALPPRAGCKKYSRGESIYDAFVIYSSQNEDWVRNELVKNLEEGVPRFHL
CLHYRDFIPGVAIAANIIQEGFHKSRKVIVVVSRHFIQSRWCIFEYEIAQTWQFLSSRSGIIFIVLE
KVEKSLLRQQVELYRLLSRNTYLEWEDNPLGRHIFWRRLKNALLDGKASNPEQTAEEEQETAT
WT.

### Experimental

A series of novel CARs containing toll-interleukin receptor (TIR) intracellular signalling domain of TLR4 that promoted cDC1s activation upon antigen recognition in the absence of adjuvants was generated. Additional experiments with the inclusion with various combinations of other intracellular domains were also carried out. Controlled experiments without the TIR domain helped informed the role this domain had on the invention.

The results show that the expression of an anti-HER2 CAR on conventional type I (cDC1s) with the CAR containing the TIR domain, increased their targeting to HER2⁺ tumour cells, which resulted in increased acquisition of tumour proteins, and most importantly, the presentation of other tumour antigens to CD8+ T cells.

### Example 1 cDC1s Control Tumour Growth Via Enhancing T Cell Responses

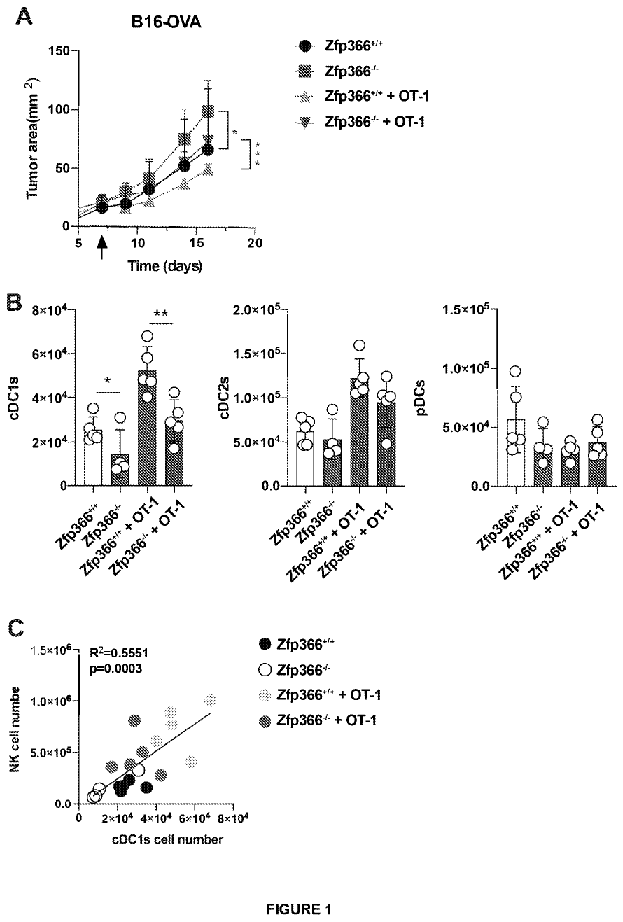

It was previously reported that deficiency of DC-SCRIPT (a protein encoded by an 8 kb mRNA preferentially expressed in DC; V Triantis et al., (2006) J Immunol 15; 176(2):1081) crippled the development and antigen presentation of cDCs (S Zhang et al., (2021) Sci Immunol 2; 6 (58))). To determine whether the impaired antigen presenting function of cDC1s would impact the efficiency of adoptive T cell therapy, Ly5.2+ WT and Zfp366^{-/-} mice were challenged with the B16-F10 melanoma cells expressing OVA antigen (B16-OVA). Ly5.1+ CD8+ OT-1 T cells were subsequently injected intravenously into the tumour challenged mice at day 7. Adoptive T cell transfer led to a remarkable reduction in tumour burden in WT mice compared with the Zfp366^{-/-} mice (FIG. 1A). The number of tumour infiltrating cDC1s was fewer in Zfp366^{-/-} mice compared with the WT mice, and the number of pDCs and cDC2s remained unchanged within the tumour (FIG. 1B). The number of Ly5.1+ OT-1 cells was also reduced in Zfp366^{-/-} mice tumour, spleen and tumour draining lymph node (LN) (data not shown), suggesting that the impaired cDC1s reduced the efficiency of adoptive T cell therapy. Intriguingly, the tumour-infiltrating T cells expressed a high level of PD-1 with or without adoptive T cell therapy, which suggested that the function of T cells may remain suppressed within the tumours. The number of CD4+ T cells, Tregs, macrophages, monocytes, and granulocytes did not change, however the number of tumour infiltrating NK cells was decreased in the Zfp366^{-/-} group after OT-1 treatment compared with the WT group. NK cell numbers positively correlated with cDC1 numbers within the tumour microenvironment (FIG. 1C).

### Example 2 cDC1 are Required for Anti-Tumour Immunity

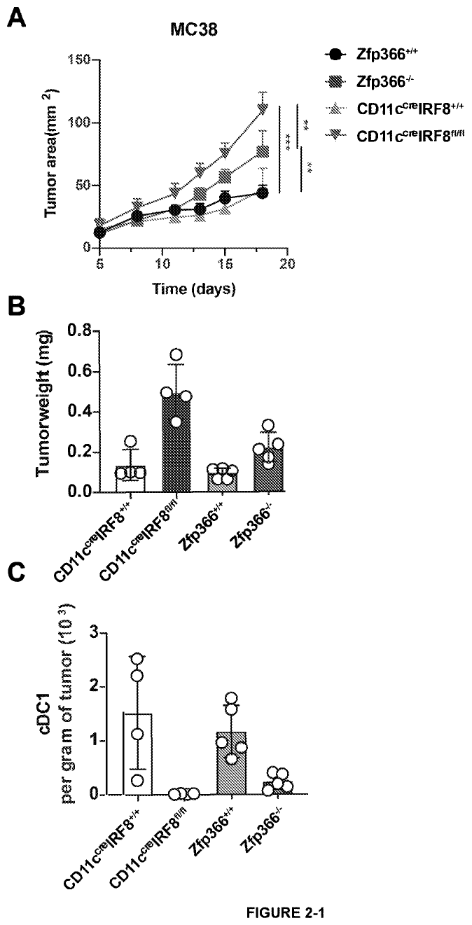

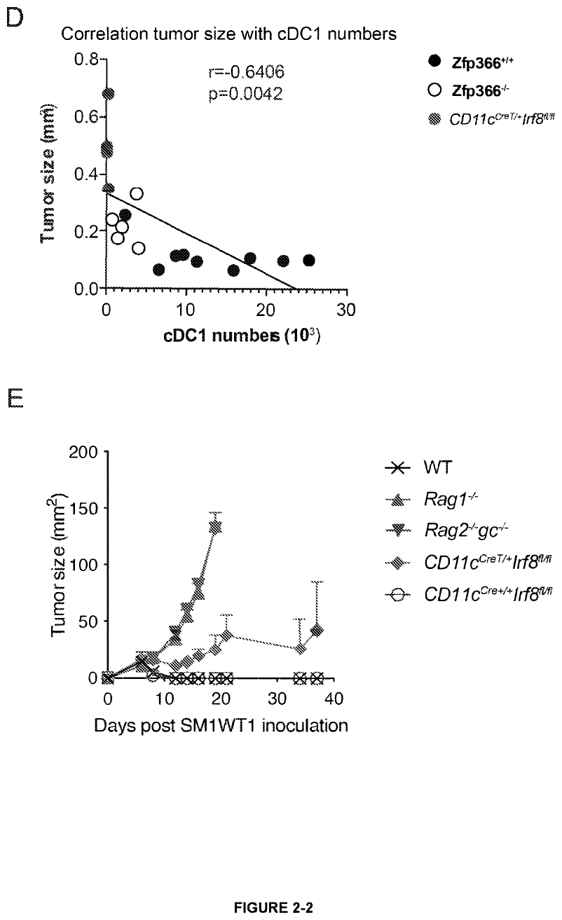

To investigate the impact of cDC1 on tumour growth, MC38 colorectal cancer cells were injected into WT, Zfp366^{-/-}, CD11c^{cre}Irf8^{fl/fl} and CD11c^{cre}Irf8^{+/+} mice. The tumours grew the fastest in CD11c^{cre}Irf8^{fl/fl} mice and Zfp366^{-/-} mouse compared with the WT control group (FIG. 2A, B). The greatest tumour burden was observed in CD11c^{cre}Irf8^{fl/fl} mice which had a complete loss of cDC1s (FIG. 2C). The number of tumour-infiltrating cDC1s was representative of the negative correlation with the tumour size (FIG. 2D). These data showed that cDC1s are critical in controlling the tumour burden. The number of tumour-infiltrating cDC1s had a positive correlation with the number of tumour-infiltrating CD8+ T cells and NK cells, but not CD4+ T cells demonstrating that the tumour-infiltrating DCs exhibited a cross-talk with CD8+ T cells, compared with CD4+ T cells, which confirmed the antigen cross-presentation ability of cDCs predominantly impacting CD8+ T cells.

Furthermore, when mice were challenged with SM1WT1 melanoma, CD11c^{cre}Irf8^{fl/fl} mice could suppress tumour growth at an early stage, but were unable to control tumour growth in the long term suggesting that cDC1s are necessary for controlling tumour progression at both an early and late stage of melanoma progression in this model (FIG. 2E). This was compared to Rag1^{-/-} mice and Rag2^{-/-}γC^{-/-} mice that lacked lymphocytes and thus tumour control, and WT and CD11c^{cre}Irf8^{+/+} mice that cleared the tumour.

### Example 3 CAR Engineered cDC1s Exhibit Enhanced Phagocytic Activity Against Targeted Antigen Positive Tumours

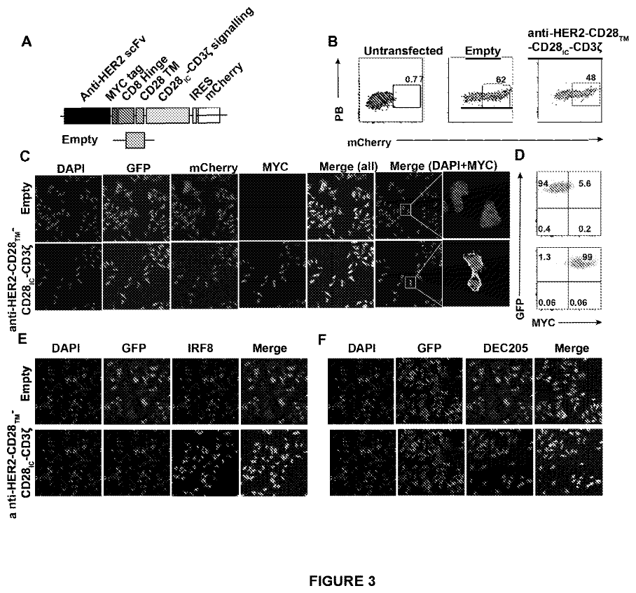

The inventors hypothesised that the expression of CARs in cDC1s could enhance their antigen recognition, uptake and presentation ability and thus to be employed as a targeted anti-tumour therapy. To examine the potential for CAR-mediated redirection of cDC1s phagocytosis, the inventors transduced the murine cDC1 cell line (mutuDCs) (S A Fuertes Marraco et al., (2012) Fontiers in Immunology 3:331) with a second-generation anti-HER2 CAR which encodes the anti-human HER2 single chain variable fragment (ScFv), a transmembrane domain and the CD28 and CD3ζ intracellular domains (FIG. 3A).

The anti-HER2 CAR was engineered into the pMSCV-mCherry retroviral vector that was transduced into mutuDCs with high efficiency and expressed CAR. The mCherry positive cDC1s were purified and could be cultured over two months maintaining a high mCherry signal. As the CAR construct encoded for a Myc tag, the inventors confirmed that mCherry transduced cDC1s have cell-surface expression of the anti-HER2 ScFv (FIG. 3B). The inventors then tested whether the expression of the CAR was detrimental to the identity of the cDC1s by measuring the expression of interferon regulatory factor 8 (IRF8) transcription or cell surface marker DEC205, key markers of cDC1s (FIG. 3C, D). This analysis revealed normal expression of these markers compared to WT mutuDCs, indicating that the transduced cells maintained their cDC1 identity (FIG. 3E, F). These data also demonstrated that cDC1s can be efficiently transduced and express the CAR.

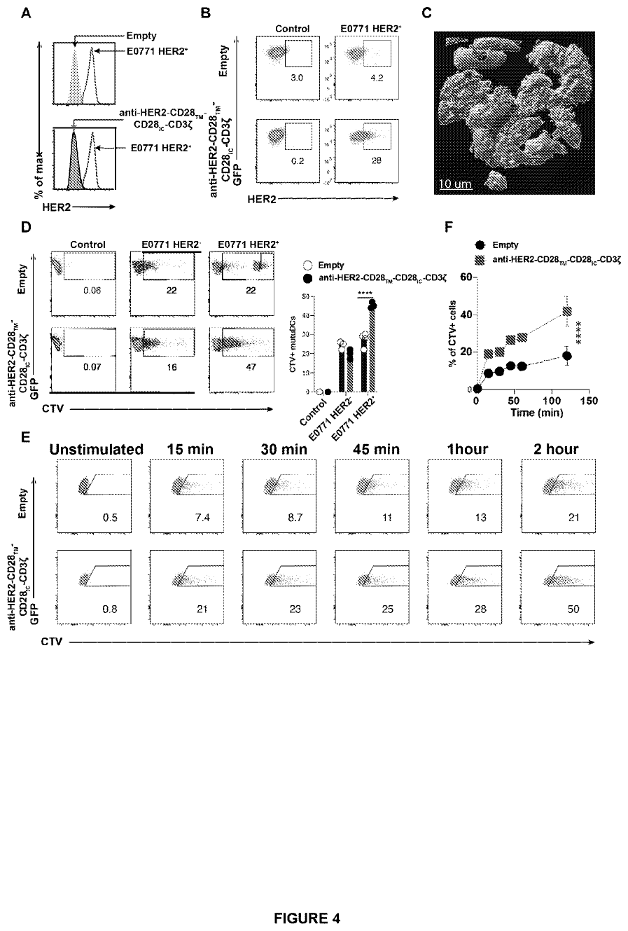

To test the function of the anti-HER2-CD28_{TM}-CD28_{IC}-CD3ζ CAR-cDC1s, the inventors measured the uptake of apoptotic bodies released from the human HER2⁺ E0771 breast cancer cell line. The HER2 expression in E0771 was confirmed via flow cytometry (FIG. 4A). Intriguingly, in contrast to cDC1s transduced with the empty CAR, the inventors found that about 28% of cDC1s transduced with anti-HER2-CD28_{TM}-CD28_{IC}-CD3ζ CAR were positive for HER2 following co-culture with the apoptotic HER2⁺ E0771 tumour cells for 2 hours, but not in the cDC1s transduced with empty vector (FIG. 4B) suggesting that CAR expression facilitated the take up of the tumour associated antigen more efficiently. Apoptotic HER2+E0771 tumour cells were labelled with CTV and cocultured with cDC1s transduced with anti-HER2-CD28_{TM}-CD28_{IC}-CD3ζ CAR as per the methods herein. Confocal imaging confirmed that the cDC1s captured the apoptotic bodies released from the HER2⁺ E0771 tumour cells (FIG. 4C). CTV density was measured via flow cytometry confirming that transduction of anti-HER2-CD28_{TM}-CD28_{IC}-CD3 CAR can promote cDC1s capture of HER2⁺ tumour associated antigen (FIG. 4D). When analysed in a timecourse, expression of anti-HER2-CD28_{TM}-CD28_{IC}-CD3ζ CAR in cDC1s occurred from at least 15 mins of coculture (FIGS. 4E and F).

This suggested that the expression of the CAR on the cell surface of the DCs may enable them to increase their uptake of tumour derived apoptotic bodies. Furthermore, anti-HER2-CD28_{TM}-CD28_{IC}-CD3ζ CAR expression can promote cDC1s recognition of HER2⁺ tumours, ultimately leading to the capture of a large array of tumour-associated antigens.

### Example 4 CAR+ cDC1s Promote cDC1s Activation after Encountering Tumour Cell-Associated Antigen

After receiving stimulation signals, cDC1s activate and express costimulatory factors, cytokines, chemokines and chemokine receptors, the best characterised of which are CD80 and CD86, which are necessary for antigen presentation to T cells. To evaluate whether CAR expression in cDC1s initiates activation of cDC1s after encountering tumour associated antigen the inventors stimulated the empty vector or anti-HER2-CD28_{TM}-CD28_{IC}-CD3ζ CAR transduced cDC1s with apoptotic HER2⁺ E0771 tumour cells or the TLR ligands such as LPS or CpG for 6 hours.

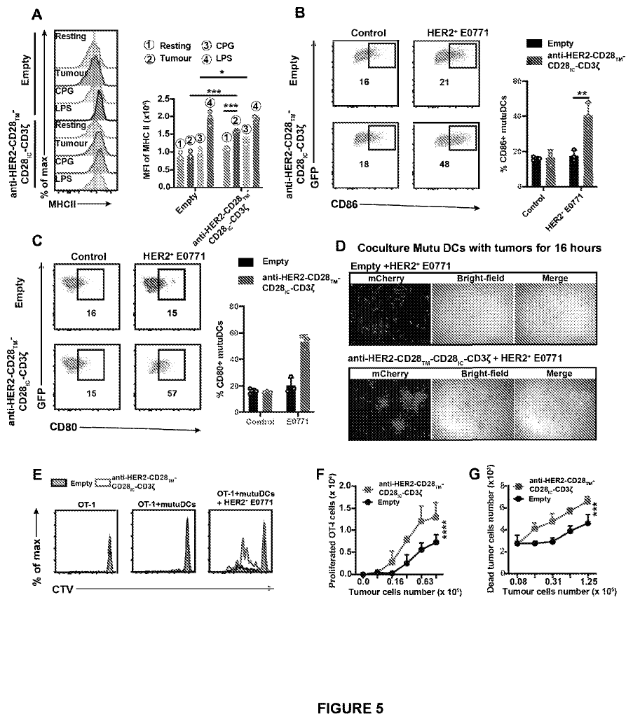

Both empty vector or anti-HER2-CD28_{TM}-CD28_{IC}-CD3ζ CAR transduced cDC1s responded to the TLR ligands LPS or CpG by increasing their surface expression of MHC-II (FIG. 5A), a canonical marker of DC maturation. The stimulation was then extended for 16 hours to determine the expression of costimulatory factors CD80 and CD86 and the results show that anti-HER2-CD28_{TM}-CD28_{IC}-CD3ζ CAR transduced cDC1s could be activated following encountering apoptotic HER2⁺ E0771 tumour cells, but the empty vector control cDC1s remained CD80/86 low (FIGS. 5B and C). Morphologically, anti-HER2-CD28_{TM}-CD28_{IC}-CD3ζ CAR transduced cDC1s tended to form clusters post-stimulation with the apoptotic HER2⁺ E0771 tumour cells, which is typical of activated cDC1s (FIG. 5D). These data demonstrate that CAR expression in cDC1s can promote cDC1 activation upon encountering tumour associated antigen.

Most tumour specific antigens are unidentified and thus are not directly amenable to a CAR-T strategy. However, the capacity of cDC1s to acquire many potential unidentified tumour associated antigens by phagocytosis and cross present them to CD8+ T cells may overcome this limitation. To investigate whether CAR expression in cDC1s can assist these cells in acquiring an independent tumour antigen, the inventors loaded the HER2⁺ E0771 cells with OVA and incubated them with the anti-HER2 CAR expressing mutuDCs in the presence of OT-I cells (transgenic CD8+ T cells that are specific for an OVA peptide). As observed earlier, anti-HER2 CAR expression in the cDC1s enhanced the cDC1s uptake of the HER2⁺ tumour-associated antigen together with OVA, which resulted in the efficient presentation of OVA derived peptides to OT-I T cells and their proliferation (FIG. 5E). Empty CAR mutuDCs had limited capacity to prime CD8 T cells in this experimental setting. In addition, anti-HER2 CAR expression in cDC1s increased their sensitivity, compared to empty vector control, across of large range of tumour cell numbers, as measured by OT-I proliferation and tumour killing (FIG. 5F, G).

Together, these data show that CAR expression on cDC1 may result in the production of a broader CD8+ T cell response via the cross-presentation of an array of tumour cell associated antigens.

### Example 5 Bone Marrow-Derived CAR cDC1s Improve their Phagocytosis in Response to Tumour-Associated Antigen

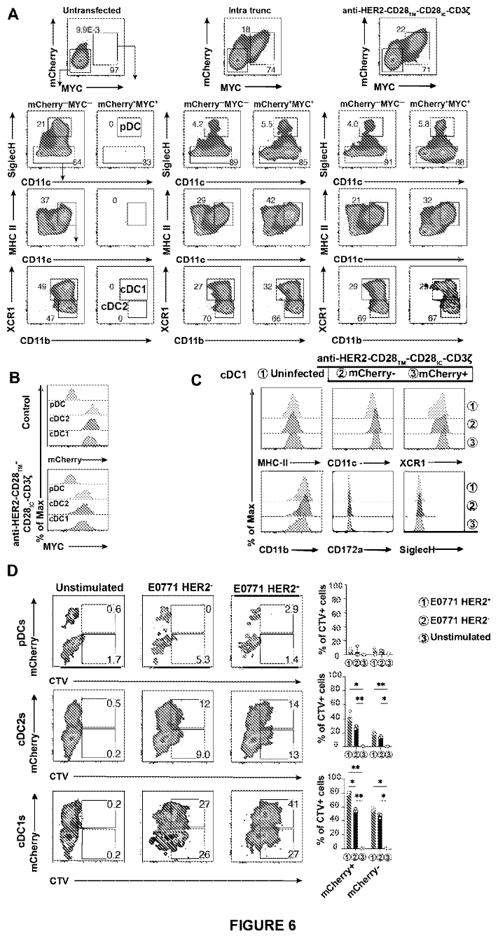

To boost the anti-tumour immune response in patients, autologous cDC1s need to be used in clinical settings. It is important to know whether bone marrow derived dendritic cells (BMDC) could be efficiently transduced by the anti-HER2-CD28_{TM}-CD28_{IC}-CD3ζ CAR retrovirus, if the signalling tail impacts cDC1 development in vitro and whether the resulting BMDCs expressed the CAR appropriately on their cell surface. To investigate these issues, the inventors cultured mouse BM cells in presence of FLT3L and transduced these cells with the standard anti-HER2-CD28_{TM}-CD28_{IC}-CD3ζ CAR or an intracellular domain truncated CAR variant (comprising a truncated CD28 signalling domain) and used them at day 4. The differentiation of DCs was examined at day 7 by flow cytometry. The Myc tag co-expressed with mCherry in BMDCs, suggesting that the anti-HER2 CAR can be transduced into the BMDCs and be expressed on the cell surface (FIG. 6A).

Both mCherry+Myc+ and mCherry−Myc− BMDCs produced pDCs, cDC1s and cDC2s with similar ratios, as measured by anti-MYC Ab staining, (FIG. 6A). The CAR transduction and expression levels were similar within DC subtypes (FIG. 6B). More importantly, the expression of typical DC markers was similar in anti-HER2-CD28_{TM}-CD28_{IC}-CD3ζ CAR expressed cDC1s compared to cDC1s expressing the truncated CAR (FIG. 6C). These data indicated that BM-derived cDC1s are formed normally in the presence of a functional CAR.

To investigate whether anti-HER2-CD28_{TM}-CD28_{IC}-CD3ζ CAR expression could enhance the tumour-associated antigen uptake, the inventors co-cultured BMDCs with CTV labelled HER2⁺ or HER2− E0771 apoptotic tumour cells for 2 hours and then assessed the antigen uptake by flow cytometry. Consistent with an earlier report (S Zhang et al., (2021) Sci Immunol 2; 6 (58) eabf4432. doi: 10.1126/sciimmunol.abf4432), cDC1s are superior in their capacity to phagocytose cell-associated antigen when compared to pDCs or cDC2s. anti-HER2-CD28_{TM}-CD28_{IC}-CD3ζ CAR expression in cDC1s enhanced their uptake of HER2+ tumour-associated antigens, but not the HER2⁻ tumour-associated antigens (FIG. 6D). This data further demonstrated that anti-HER2-CD28_{TM}-CD28_{IC}-CD3ζ CAR cDC1s can also enhance the phagocytic ability of BM-derived cDC1s in a target specific manner.

### Example 6 Transduction of Novel TIR4 Containing CARs Generates Supercharged cDC1s

TLR4 is a family member of the pattern-recognition receptor that defends against microbial infection by interacting with lipopolysaccharide (LPS). TLR4 contains a cytosolic Toll/interleukin-1 receptor (TIR) domain, and signal transduction is initiated by dimerization of the TIR domain after LPS binding which leads to the recruitment of adaptor proteins triggering downstream activation of inflammation and anti-pathogen responses. TLR4 signalling promotes cDC1 maturation and favours the production of Th1 cytokine via activating NF-kB, AP-1 and interferon regulatory (IRF) signals in the MyD88-dependent and TRIF-mediated pathway to them facilitate the generation of cytotoxic T lymphocytes (CTLs) and Th1 cells (T Ve et al., (2012) Curr Drugs Targets 13:1360-1374; N J Gay et al., (2014) Nature reviews. Immunology 14:546-558). Therefore, the inventors proposed that TLR4 signalling could potentiate the anti-tumour efficiency of CAR-cDC1s.

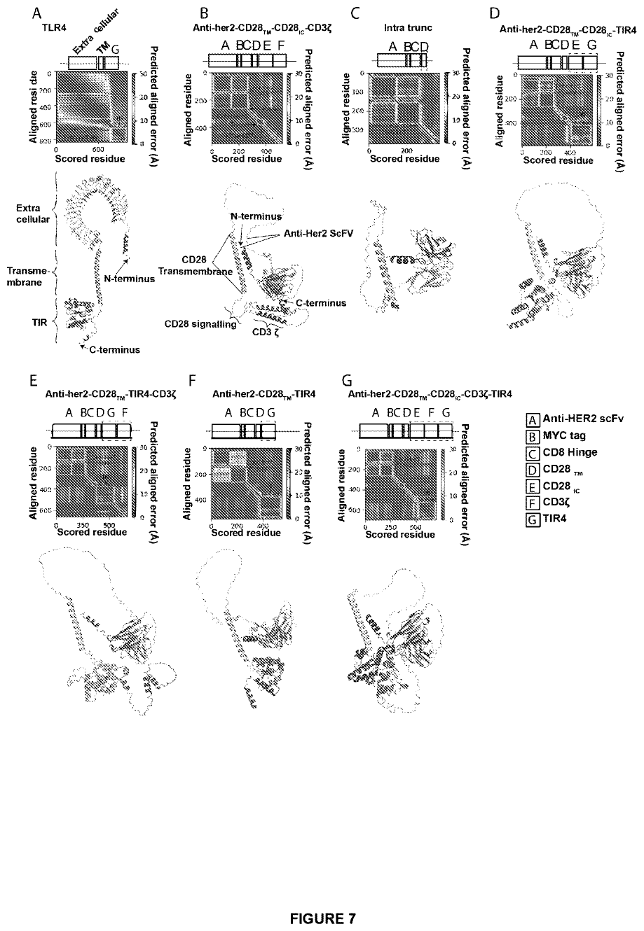

The inventors hypothesised that replacing the CD28_{IC}-CD3ζ intracellular domain of CAR with the TIR4 domain of TLR4 could initiate inflammatory responses with DCs after encountering CAR-targeted tumour antigen. To test this the inventors designed four types of new CARs targeting human HER2 by using the intracellular signalling (TIR) domain of toll-like receptor 4 (TLR4), to replace the CD28 and/or CD3ζ signalling domains or by appending to the C-terminus of the CD3ζ domain. The de novo protein structure of the new CAR was predicted by AlphaFold 2 with high confidence (>90%) for the functional domain. The TIR domain adopts a flavodoxin-like fold, featuring a central 5 stranded parallel β sheet that is surrounded by 5 α helices on both sides of the sheet which are connected by loops (FIG. 7A). The extracellular anti-HER2 ScFv domain form several anti parallel β sheets and linked with the α helix CD28_{TM} domain by the non-regular coil of CD8 hinge (FIG. 7B). The intracellular CD28 signalling domain forms a nonregular coil and appended to CD3ζ which forms a triple helix. To evaluate the necessity of the intracellular domain of the anti-HER2 CAR for normal function in cDC1s the intracellular domain was truncated in the control CAR.

The α-helix structure of the transmembrane domain was maintained after truncating the intracellular domain (FIG. 7C). This feature is critical for the truncated CAR to be expressed on the cell surface. Replacing the CD28 and/or CD3ζ domain with the TIR domain is predicted to preserve its flavodoxin-like fold structure (FIG. 7D-G) which is critical to retain its signalling function. Furthermore, the extracellular anti-HER2-ScFV structure was well preserved after modifying the intracellular domain (FIG. 7D-G) which is less likely to impact the affinity of anti-HER2-ScFV to the human HER2 antigen.

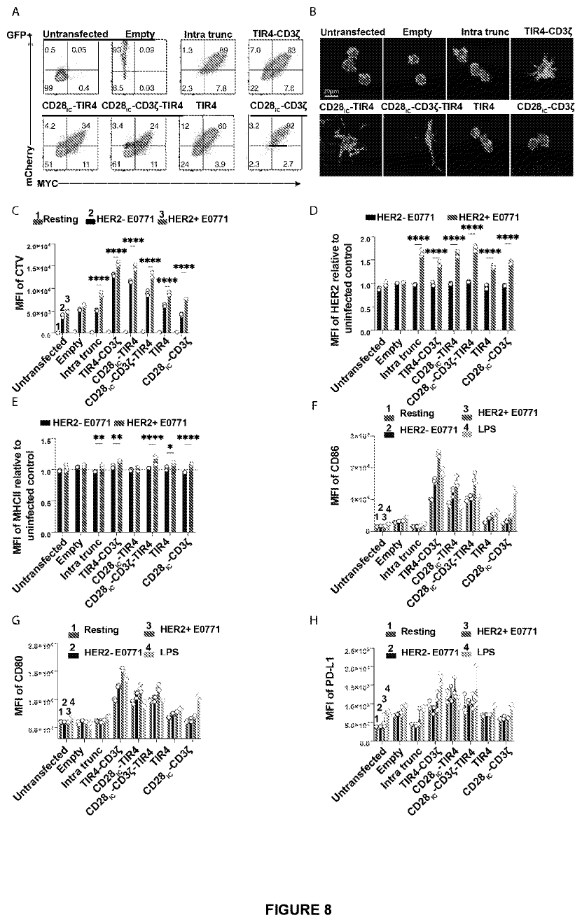

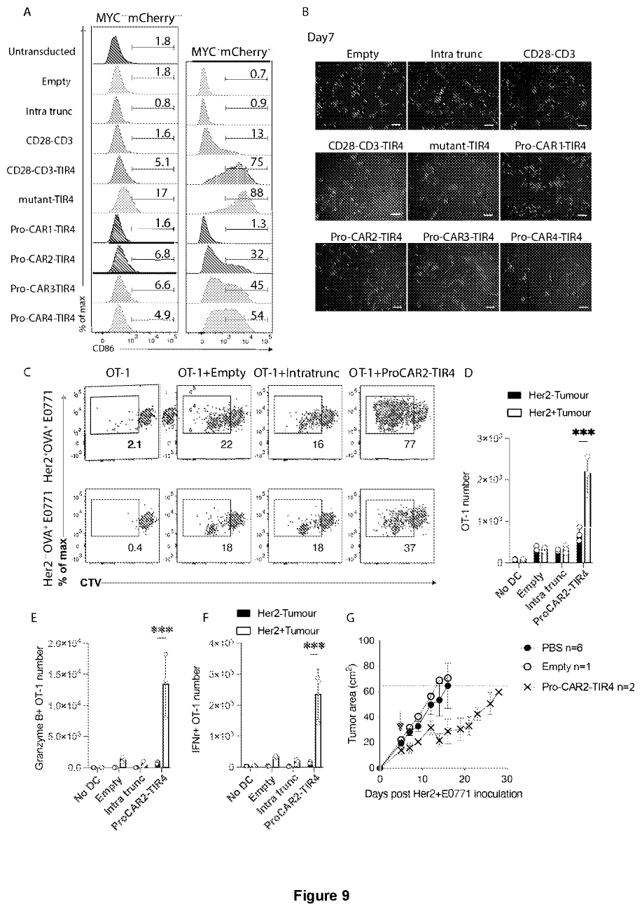

To test whether the four different TIR4 CARs could be expressed on the cDC1 cell surface, the CAR constructs were transduced into mutuDCs. Anti-Myc staining of mutuDCs transduced with the anti-HER2 CAR showed efficient and sustained cell-surface expression of the anti-HER2 CAR (FIG. 8A). Morphologically, anti-HER2-CD28_{TM}-TIR4-CD3ζ CAR, anti-HER2-CD28_{TM}-CD28_{IC}-TIR4 CAR or anti-HER2-CD28_{TM}-CD28_{IC}-CD3ζ-TIR4 CAR transduced cDC1s showed enlarged dendrites without any stimulation signal (FIG. 8B), but this feature was not observed in the empty vector intracellular domain truncated CAR, TIR4 only CAR or CD28_{IC}-CD3ζ CAR transduced cDC1s.

Phagocytosis experiments revealed cDC1s transduced with the anti-HER2 CAR readily took up the HER2⁺ tumour associated antigen, especially the anti-HER2-CD28_{TM}-TIR4-CD3ζ CAR, anti-HER2-CD28_{TM}-CD28_{IC}-TIR4 CAR or anti-HER2-CD28_{TM}-CD28_{IC}-CD3ζ-TIR4 CAR transduced cDC1s (FIG. 8C). This transfer was specific as the HER2 antigen only transferred from HER2⁺ tumour cells to the anti-HER2 CAR transduced cDC1s, but not to the uninfected or empty vector CAR transduced cDC1s (FIG. 8D). The inventors observed only a slightly increased level of expression of MHC-II on HER2⁺ E0771 stimulated cDC1s (FIG. 8E). The inventors examined the capacity CAR expressing cDC1s to respond to HER2⁺ tumour cells and TLR4 agonists. All the cDC1s transduced with an intracellular domain containing anti-HER2 CAR responded to the HER2⁺ tumour-specific antigen by increasing their surface expression of CD80 and CD86, canonical markers of DC maturation (FIGS. 8F and G). The CD80 and CD86 expression levels were comparable to those induced by lipopolysaccharride (LPS) stimulation. Consistent with the morphological changes in anti-HER2-CD28_{TM}-TIR4-CD3ζ CAR, anti-HER2-CD28_{TM}-CD28_{IC}-TIR4 CAR or anti-HER2-CD28_{TM}-CD28_{IC}-CD3ζ-TIR4 CAR transduced cDC1s, CD80 and CD86 also expressed more highly in the unstimulated cells compared with the other treatment groups. PD-L1 expression in the anti-HER2 CAR transduced cDC1s remained unchanged following stimulation with HER2⁻ or HER2⁺ tumour cells (FIG. 8H).

These data demonstrated that the anti-HER2-CD28_{TM}-TIR4-CD3ζ CAR, anti-HER2-CD28_{TM}-CD28_{IC}-TIR4 CAR and anti-HER2-CD28_{TM}-CD28_{IC}-CD3ζ-TIR4 CAR of the invention; 1) are expressed on the cDC1 cell surface; 2) have endowed cDC1s to phagocytose HER2⁺ tumour-specific antigen with high efficiency; 3) promote a TLR4 dependent cDC1 activation upon cognate recognition of their target.

### Example 7 the Presence of Co-Stimulatory Factors are Required for the Activation of Anti-HER2-CD28_{TM}-CD28_{IC}-CD3ζ-TIR4 CAR

After incorporating the TIR4 domain into the distal part of the signaling domain of the anti-HER2-CD28_{TM}-CD28_{IC}-CD3ζ CAR, the inventors observed the autonomous activation of TIR4 in an antigen-independent manner. Activation of TLR4 in vivo typically necessitates the formation of homodimers upon binding with LPS. The hinge-CD28_{TM} region contains a cysteine residue that drives disulfide-linked receptor homodimerization. Additionally, the CD28_{TM} region harbors a highly conserved polar YxxxxT motif crucial for optimal dimerization and recruitment of supplementary endogenous costimulatory signals. However, adding TIR4 alone after the CD28_{TM} domain did not result in the (auto) activation of the TIR4 domain (FIG. 8B), indicating the significance of the CD28/CD3ζ signalling distal domains in promoting TIR4 activation.

The inventors thus explored the possibility of designing different motifs into the transmembrane domain that would prevent oligomerisation in the absence of the ligand and thus prevent TLR4 autoactivation. To this end the inventors mutated the cysteine residue in the CD8a hinge region to alanine (mutant-TIR4) and in a further experiment changed both the hinge and substituted the YSLLVT motif within the transmembrane domain with a different motif, designed to promote a dimeric state (labelled Pro-CAR2-TIR4). The design of this synthetic transmembrane domain was informed by work previously published in WO2021/229581 and Elazar, et al. eLife 2022; 11. Changing the transmembrane domain in addition to the single mutation in the hinge region significantly reduced the autoactivation compared to the earlier CAR, anti-HER2-CD28_{TM}-CD28_{IC}-CD3ζ-TIR4 CAR, but mutating only the cysteine residue in the hinge region did not have the same effect on autoactivation (FIGS. 9A and B). These findings suggest that the activation of the TIR4 domain may result from a synergistic effect between the transmembrane domain and the CD28 intracellular signalling domain.

To explore whether the use of this synthetic, designed transmembrane domain, (labelled Pro-CAR2-TIR4) in cDC1s could allow these cells to be activation in a tumour antigen dependent manner, the inventors loaded HER2⁺ E0771 cells with OVA and incubated them with mutuDCs expressing the anti-HER2-Pro-CAR2-CD28_{IC}-CD3ζ-TIR4 CAR, along with OT-I cells. Use of OVA as a neoantigen and measuring the capacity of the various embodiments of this invention to stimulate the proliferation of OT-1 cells in response to cognate recognition of HER2+ E0771 tumour cells, and ultimately promote the killing of OVA-loaded tumour cells, is a measure of antigen cross-presentation and anti-tumour immunity.

The expression of anti-HER2-Pro-CAR2-CD28_{IC}-CD3ζ-TIR4 CAR in cDC1s augmented the uptake of HER2⁺ tumour-associated antigen along with OVA, resulting in efficient presentation of OVA-derived peptides to OT-I T cells and subsequent proliferation (FIGS. 9C and D). The population of Granzyme B+ cytotoxic T cells and IFN-γ-producing T cells was significantly enriched in the presence of anti-HER2-Pro-CAR2-CD28_{IC}-CD3ζ-TIR4 CAR-expressing cDC1s. Adoptive transfer of anti-HER2-Pro-CAR2-CD28_{IC}-CD3ζ-TIR4 CAR-expressing cDC1s into HER2⁺ E0771 tumor-challenged human HER2 transgenic mice suppressed tumour progression, indicating the therapeutic potential of the CAR-DC1 approach.

In summary, DCs transduced with the CAR whose intracellular domain only contains the TIR domain failed to generate a matured branching morphology which is consistent with the findings in WO 2021/1277024. The anti-HER2-CD28_{TM}-TIR4 CAR transduced DCs were able to bind to HER2⁺ tumour associated antigen but expression of DC maturation markers CD86 and CD80 did not change significantly on anti-HER2-TIR4 CAR transduced cDC1s before or after stimulating with HER2⁺ tumour antigens compared with the empty CAR or intracellular domain truncated CAR transduced cDC1s.

The matured branching morphology and the increased expression of maturation markers were observed within the cDC1s transduced with the CAR containing TIR4 and CD28_{IC}, the CAR containing TIR4 and CD3ζ and the CAR containing TIR4, CD28_{IC} and CD3ζ. These experiments indicate that the most effective CAR sequences include both a TIR4 domain and an additional costimulatory activation domain (eg CD3ζ, CD28_{IC} or both). The inventors also checked the transcriptomic changes of anti-HER2-CD28_{TM}-TIR4 CAR transduced cDC1s by RNA sequencing. The transcriptomic changes of anti-HER2-CD28_{TM}-TIR4 CAR transduced cDC1s are very close to the intracellular domain truncated CAR transduced cDC1s (data not shown). The differential gene expression number between anti-HER2-CD28_{TM}-TIR4 CAR transduced cDC1s and intracellular domain truncated CAR transduced cDC1s (401 Up, and 37 down) is much less compared with the anti-HER2-CD28_{TM}-CD28_{IC}-TIR4 CAR vs intracellular domain truncated CAR transduced cDC1s (1344 Up, and 592 down) or anti-HER2-CD28_{TM}-CD28_{IC}-CD3ζ-TIR4 CAR transduced cDC1s VS intracellular domain truncated CAR transduced cDC1s (1817 UP, and 855 down). This data demonstrated that the TIR domain alone was not be able to induce the activation of cDC1s after HER2⁺ tumour antigen stimulation and instead required additional co-stimulatory domains such as CD3ζ, CD28_{IC} or both.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

1. A chimeric antigen receptor (CAR) construct comprising:
(i) an antigen-binding domain having binding affinity to a tumour cell antigen;
(ii) a transmembrane domain (TM); and
(iii) an intracellular domain (IC) comprising a toll-interleukin receptor (TIR) intracellular signalling domain; and a costimulatory signalling domain selected from a CD3 signalling domain, CD28 signalling domain and a combined CD28 and CD3 signalling domain,
wherein the CAR construct is capable of being expressed and is functional in a dendritic cell.

2. A modified dendritic cell comprising the chimeric antigen receptor (CAR) construct of claim 1 or comprising one or more nucleic acid sequences encoding the CAR construct of claim 1 wherein the modified dendritic cell is capable of antigen cross-presentation to T cells, an adaptive immune response, or activation of antitumour T cells.

3. The CAR construct according to claim 1, wherein the TIR signalling domain is situated after the costimulatory signally domain at the C-terminal end of the CAR.

4. The CAR construct according to claim 1, wherein the transmembrane domain is a CD28 transmembrane domain or is a synthetic transmembrane domain.

5. The modified dendritic cell according to claim 2, wherein the transmembrane domain is a CD28 transmembrane domain or a synthetic transmembrane domain.

6. The modified dendritic cell according to claim 5 wherein the synthetic transmembrane domain is designed to promote olimerisation, increase specificity, suppress constitutive signalling or a combination of any these properties.

7. The modified dendritic cell according to claim 2, wherein the dendritic cell is a conventional type I (cDC1) cell.

8. The CAR construct according to claim 1, wherein the CAR further comprises a CD8 hinge sequence.

9. (canceled)

10. (canceled)

11. The CAR construct according to claim 1, wherein the TIR intracellular signalling domain is selected from Toll-like receptor 4, Toll-like receptor 3, Toll-like receptor 9, Toll-like receptor 11 and Toll-like receptor 13.

12. The CAR construct according to claim 11, wherein the TIR intracellular signalling domain is a Toll-like receptor 4 (TIR4).

13. The CAR construct according to claim 12, wherein the TIR4 domain comprises or consists of the sequence
(SEQ ID NO: 6)
AGCKKYSRGESIYDAFVIYSSQNEDWVRNELVKNLEEGVPRFHLCLHYR
DFIPGVAIAANIIQEGFHKSRKVIVVVSRHFIQSRWCIFEYEIAQTWQF
LSSRSGIIFIVLEKVEKSLLRQQVELYRLLSRNTYLEWEDNPLGRHIFW
RRLKNALLDGKASNPEQTAEEEQETATWT.

14. The CAR construct according to claim 1, wherein the CAR construct comprises or consists of the sequence of SEQ ID NO:7, SEQ ID NO:8 or SEQ ID NO:9.

15. The CAR construct of claim 1, wherein the antigen binding domain comprises an antibody, an antibody fragment or antigen binding fragment thereof.

16. (canceled)

17. The CAR construct of claim 1, wherein the antigen binding domain binds to HER2 antigen.

18. The CAR construct according to claim 17, wherein the antigen binding domain is an scFv.

19. The CAR construct of claim 1, wherein the CAR transmembrane domain (TM) and intracellular domain comprises or consists of the sequence of SEQ ID NO:7, SEQ ID NO:8, or SEQ ID NO:9.

20. (canceled)

21. A nucleic acid sequence encoding the CAR of claim 19.

22. (canceled)

23. A pharmaceutical composition comprising the modified dendritic cell according to claim 2.

24. A method of stimulating an adaptive immune response in a subject having cancer comprising: administering to the subject a therapeutically effective amount of the modified dendritic cell according to claim 2.

25. The method according to claim 24, wherein the subject is administered a modified dendritic cell comprising a CAR having the transmembrane domain (TM) and intracellular domain sequence set forth in any one of SEQ ID NO:7, SEQ ID NO:8, or SEQ ID NO:9.

26. (canceled)

27. The method according to claim 24, wherein the method induces phagocytosis of apoptotic bodies released from cancer cells in the subject.

28. The method according to claim 24, wherein the modified dendritic cell induces a tumour-eliminating immune response.

29. A method for making a chimeric antigen receptor containing dendritic cell (CAR-DC) population, comprising:
(i) providing a population of cells from a subject;
(ii) culturing the population of cells in a medium comprising an FMS-like tyrosine kinase 3 (Flt3) agonist for at least one day;
(iii) introducing into the population of cells a chimeric antigen receptor (CAR) comprising an antibody or antigen-binding fragment thereof having binding affinity to a tumour cell antigen, a transmembrane domain (TM), and an intracellular domain (IC) comprising a toll-interleukin receptor (TIR) intracellular signalling domain and a costimulatory signalling domain selected from a CD3 signalling domain, CD28 signalling domain and a combined CD28 and CD3 signalling domain, wherein the TIR signalling domain is situated after the costimulatory signally domain at the C-terminal end of the CAR; and
(iv) culturing the cells from (iii) in a medium comprising an FMS-like tyrosine kinase 3 (Flt3) agonist for an amount of time sufficient to form a modified dendritic cell.

30. The method according to claim 29, wherein the population of cells is selected from mononuclear or stem cells from circulation, cord or bone marrow.

31. The method according to claim 29, wherein the dendritic cell is an autologous or heterologous conventional type I dendritic cell (cDC1).
